(19) United States
(12) Patent Application Publication
(10) Pub. No.: US 2009/0306728 A1
(43) Pub. Date: Dec. 10, 2009

(54) METHODS AND APPARATUS FOR STIMULATING AND/OR SENSING NEURONS IN A PATIENT

(75) Inventors: J. Nelson Wright, Mercer Island, WA (US); Steven C. Dimmer, Bellevue, WA (US)

Correspondence Address: PERKINS COIE LLP;PATENT-SEA, P.O. BOX 1247, SEATTLE, WA, 98111-1247, US

(73) Assignee: CALYPSO MEDICAL TECHNOLOGIES, INC., Seattle, WA (US)

(21) Appl. No.: 10/585,493

(22) PCT Filed: Jan. 12, 2005
(86) PCT No.: PCT/US2005/001070

§ 371 Date: Aug. 17, 2009

Related U.S. Application Data
(60) Provisional application No. 60/536,008, filed on Jan. 12, 2004.
(60) Provisional application No. 60/551,170, filed on Mar. 8, 2004.
(60) Provisional application No. 60/586,206, filed on Jul. 7, 2004.

(51) Int. Cl. A61N 1/36; A61N 1/08; A61N 1/05

(52) U.S. Cl. 607/3; 607/60; 607/45; 607/116

(57) ABSTRACT

Instruments and method of using instruments for implanting electrodes into a patient. The instrument can include a body configured to be implanted into a patient, an electrode contact carried by the body, and a marker carried by the body. The electrode contact has an electrically conductive surface exposed at a location along the body to sense electrical activity and/or deliver electrical stimulation to the target neural structure. The marker can include a transponder configured to be energized by a wirelessly transmitted excitation energy and to wirelessly transmit a location signal in response to the excitation energy. The instrument is tracked as it is implanted into the patient by time multiplexing the wirelessly transmitted excitation energy and the location signal such that the absolute location of the marker can be determined in real time.

### CROSS-REFERENCE TO RELATED APPLICATIONS

The present application claims the benefit of and incorporates by reference all of the following U.S. Provisional Application Nos.: 60/536,008 filed on Jan. 12, 2004; 60/551,170 filed on Mar. 8, 2004; and 60/586,209 filed on Jul. 7, 2004.

### TECHNICAL FIELD

The present invention relates to apparatus and methods for stimulating and/or sensing target neural structures in the deep brain, spine and/or other locations in a patient.

### BACKGROUND

Several mental and physical processes are controlled or influenced by neural activity in the central and peripheral nervous systems. For example, several areas of the brain appear to have distinct functions in most individuals. As a result, stimulating neurons at selected locations of the central nervous system can be used to change, induce, suppress and/or otherwise treat mental and physical functions throughout the body.

Stimulation of deep brain structures using electrical pulses, magnetic pulses and/or drugs has been studied and implemented to treat epilepsy, movement disorders, anxiety, schizophrenia, heart conditions and many other types of diseases or disorders. Several stimulation therapies use implantable devices with electrical sensors to detect the onset of an event (e.g., epilepsy or tremor), and electrical contacts to deliver electrical pulses that stimulate selected neurological structures. In a typical application, the implantable devices include a pulse generator similar to a cardiac pacemaker, a lead coupled to the pulse generator, and an elongated electrode configured to be implanted into the deep brain regions of a patient.

U.S. Pat. No. 5,713,922, which is incorporated by reference herein, discloses placing an electrode in the deep brain region of a patient proximate to the thalamus, globus pallidus and other neural structures for relief of chronic pain or to control movements. U.S. Pat. No. 5,716,377, which is also incorporated by reference herein, discloses a method for treating schizophrenia by brain stimulation and drug infusion that uses an implantable signal generator, electrode, pump and catheter to deliver drugs and electrical stimulation to deep brain locations in the patient. Other applications involve implanting electrodes or other instruments into deep brain locations for diagnostic purposes, such as mapping the neural structures or sensing neural conditions.

The electrodes, catheters and other instruments are typically implanted into the deep brain locations by cutting a burr hole in the patient's cranium and then inserting an elongated electrode into the brain until the electrical contacts are positioned at a desired location with respect to the target neural structure. More specifically, elongated electrodes are implanted by attaching a fixed reference frame to the head of the patient, imaging the patent's brain relative to the reference frame, and inserting the electrode along a selected trajectory until the contacts reach a predetermined depth. The location of the instrument relative to the target structure may be determined periodically using X-rays.

The primary problems of implanting electrodes into deep brain regions are (a) healthy neural tissue may be damaged, and (b) it is difficult to accurately position the instrument at the target neural structure. For example, if the practitioner inserts the instrument along the wrong trajectory, the instrument may pass through important neural tissue. This can damage healthy neural tissue and cause undesirable side effects. Also, if the electrodes are not accurately positioned at the target neural structure, then the stimulation may not achieve the desired results and/or it may cause undesirable collateral affects (e.g., seizures) because the electrical field is not at the optimal location. Therefore, there is a significant need to improve the accuracy with which electrodes and/or other instruments are implanted into deep brain or other neural structures.

### BRIEF DESCRIPTION OF THE DRAWINGS

FIG. 1 is a side view of an instrument configured to be implanted into a patient in accordance with an embodiment of the invention. Several features of the instrument are shown schematically in FIG. 1.

FIG. 2 is a cross-sectional view illustrating specific details of the distal end of the instrument shown in FIG. 1.

FIG. 3 is a side view of an instrument configured to be implanted into a patient in accordance with another embodiment of the invention. Several features of the instrument are shown schematically in FIG. 3.

FIG. 4A is a side view schematically illustrating an instrument, a localization system and a stimulus unit implanted in a patient in accordance with an embodiment of the invention.

FIG. 4B is a flow chart of a method for tracking an instrument in accordance with an embodiment of the invention.

FIG. 4C is a schematic side view of a system performing an embodiment of the method shown in FIG. 4B.

FIG. 5A is an isometric view of a marker for use with an instrument in accordance with an embodiment of the invention.

FIG. 5B is a cross-sectional view of the marker of FIG. 5A taken along line 5B-5B.

FIG. 5C is an illustration of a radiographic image of the marker of FIGS. 5A-B.

FIG. 6A is an isometric view of a marker for use with an instrument in accordance with another embodiment of the invention.

FIG. 6B is a cross-sectional view of the marker of FIG. 6A taken along line 6B-6B.

FIG. 7A is an isometric view of a marker for use with an instrument in accordance with another embodiment of the invention.

FIG. 7B is a cross-sectional view of the marker of FIG. 7A taken along line 7B-7B.

FIG. 8 is an isometric view of a marker for use with an instrument in accordance with another embodiment of the invention.

FIG. 9 is an isometric view of a marker for use with an instrument in accordance with yet another embodiment of the invention.

FIG. 10 is a schematic block diagram of a localization system for use in locating an instrument in accordance with an embodiment of the invention.

FIG. 11 is a schematic view of an array of coplanar source coils carrying electrical signals in a first combination of phases to generate a first excitation field.

FIG. 12 is a schematic view of an array of coplanar source coils carrying electrical signals in a second combination of phases to generate a second excitation field.

FIG. 13 is a schematic view of an array of coplanar source coils carrying electrical signals in a third combination of phases to generate a third excitation field.

FIG. 14 is a schematic view of an array of coplanar source coils illustrating a magnetic excitation field for energizing markers in a first spatial orientation.

FIG. 15 is a schematic view of an array of coplanar source coils illustrating a magnetic excitation field for energizing markers in a second spatial orientation.

FIG. 16A is an exploded isometric view showing individual components of a sensor assembly for use with a localization system to localize an instrument in accordance with an embodiment of the invention.

FIG. 16B is a top plan view of a sensing unit for use in the sensor assembly of FIG. 16A.

FIG. 17 is a schematic diagram of a preamplifier for use with the sensor assembly of FIG. 16A.

### DETAILED DESCRIPTION

### A. Overview

The present invention is directed toward apparatus and methods for implanting instruments into deep brain structures or other locations relative to the central or peripheral nervous systems. Several embodiments of the invention are directed towards instruments and systems for stimulating and/or sensor target neural structures at deep brain locations of a patient.

One embodiment of such an instrument includes a body configured to be implanted into a patient, an electrode contact carried by the body, and a marker carried by the body. The body can be an elongated structure having a biocompatible outer surface, and the body can be either rigid for implantation through tissue or flexible for implantation through the vascular system. The electrode contact has an electrically conductive surface exposed at a location along the body to sense electrical activity and/or deliver electrical stimulation to the target neural structure. The marker is located on the body relative to the electrode contact. The marker can include a transponder that receives a wirelessly transmitted excitation energy and produces a wirelessly transmitted location signal in response to the excitation energy. In operation, the electrode contact is tracked as the instrument is implanted into the brain of the patient by time multiplexing the wirelessly transmitted excitation energy and the location signal such that the absolute location of the marker can be determined in real time.

Another aspect of the invention is directed toward stimulation systems that can be implanted into the patient. One embodiment of a stimulation system in accordance with the invention comprises an implantable pulse generator, a lead configured to be coupled to the implantable pulse generator, and an electrode. The implantable pulse generator includes a housing, an energy source, and a circuit for providing electrical stimulation. The lead has a flexible dielectric cover and a conductor within the cover, and the lead is configured to be implanted within the patient. The electrode has a body configured to be implanted into the patient, an electrode contact carried by the body, and a marker carried by the body. The electrode contact is configured to be electrically coupled to the lead. The marker comprises a transponder including a circuit configured to be wirelessly powered by a pulsed excitation field, and to produce a wirelessly transmitted pulsed location signal in response to the pulsed excitation field.

Another embodiment of a system for sensing and/or stimulating a population of neurons in the nervous system comprises an electrode having a body, an electrode contact carried by the body, and marker carried by the body. The marker has a transponder that receives a wirelessly transmitted pulsed magnetic excitation field and produces a wirelessly transmitted pulsed location signal in response to the excitation field. The system further includes a field generator comprising an energy storage device, a source coil, and a switching network. The source coil produces the pulsed magnetic excitation field at a sufficient strength and for a limited duration to cause the transponder to wirelessly transmit the pulsed location signal outside of the patient. The switching network is coupled to the energy storage device and the source coil. The switching network is configured to alternately transfer (a) stored energy from the energy storage device to the source coil; and (b) energy in the source coil back to the energy storage device. In operation, the switching network actively energizes the source coil and then actively de-energizes the source coil to time multiplex the excitation field and the location signal.

Another embodiment of a system for sensing and/or stimulating a population of neurons in the nervous system of a patient comprises an electrode having a body, an electrode contact carried by the body, and a marker having a transponder. The transponder receives a wirelessly transmitted pulsed magnetic excitation field and produces a wirelessly transmitted pulsed location signal in response to the pulsed excitation field. The system further includes a sensor assembly comprising a support member and a plurality of field sensors carried by the support member for sensing the location signal from the transponder. The field sensors are at least substantially locally planar relative to one another and responsive only to field components of the location signal normal to individual field sensors. The field sensors can be arranged in an array occupying an area having a maximum dimension of approximately 100% to 300% of a predetermined sensing distance between the marker and the field sensors.

Another aspect of the invention is directed towards methods for sensing and/or stimulating a population of neurons at a selected stimulation site in the patient. One embodiment of such a method comprises implanting into the patient an electrode having an electrode contact and a marker including a transponder. The method further includes determining the location of the electrode contact and/or the marker with respect to the stimulation site by (a) wirelessly delivering a pulsed excitation signal to the transponder, (b) wirelessly transmitting a pulsed location signal from the transponder to a sensor outside the patient, (c) sensing the pulsed location signal at the sensor, and (d) calculating the absolute location of the electrode contact and/or the marker in a three-dimensional reference volume. The method can further include sensing electrical activity of the neurons and/or delivering electrical stimulation to the target neural structure.

### B. Embodiments of Instruments for Deep Brain Applications

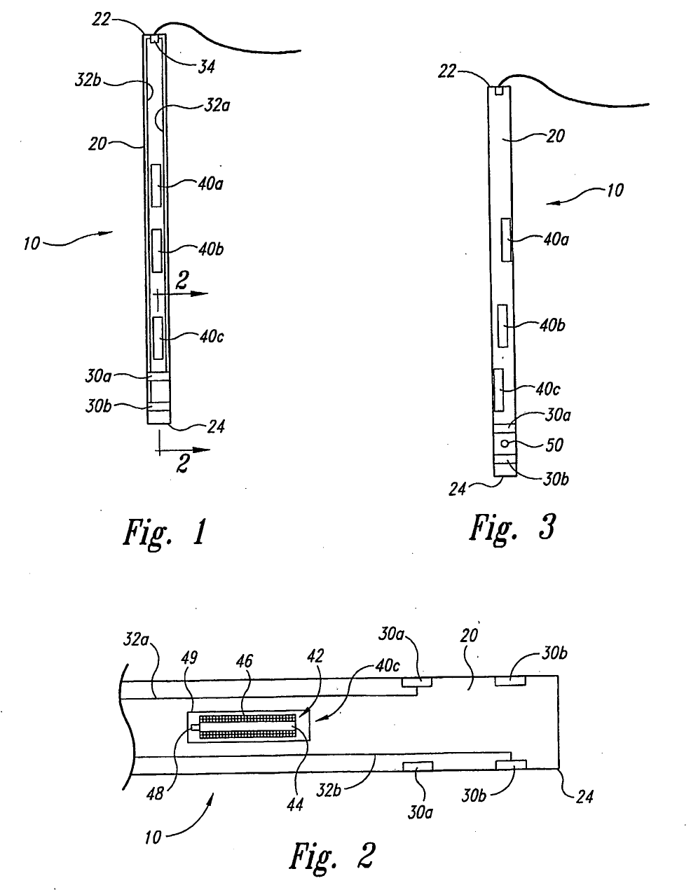

FIG. 1 is a side view of an instrument 10 for stimulating and/or sensing neural activity at a target neural structure in the nervous system of a patient. The instrument 10 includes a body 20 having a proximal end 22 and a distal end 24. In the embodiment shown in FIG. 1, the instrument 10 is an implantable electrode having individual electrode contacts 30a-b and location markers 40a-c carried by the body 20. The instrument 10 is particularly useful for sensing electrical activity at deep brain locations and/or delivering electrical stimulation to deep brain neural structures. The instrument 10, however, can also be used to sense or stimulate other sub-dural neural structures in the brain or other parts of the central nervous system (e.g., the spinal column and neck). As explained in more detail below, the markers 40 provide the absolute position and orientation of the instrument 10 in a three-dimensional reference frame to accurately guide the electrode contacts 30a-b to the target neural structure.

The body 20 shown in FIG. 1 is an elongated casing configured to be implanted into a patient. The body 20 can be substantially rigid to pass the instrument 10 through tissue for implanting the distal end 24 at deep brain locations in the patient. In other embodiments, the body 20 is flexible so that it can be inserted through the vascular system to internal locations using a catheter. The body 20 is composed of a biocompatible material, such as a suitable polymer, titanium or other materials. In the embodiment shown in FIG. 1, the body 20 has a dielectric outer surface to electrically isolate the electrode contacts 30a-b from each other. The body 20 generally has a small cross-sectional dimension to mitigate damage to neural tissue and other structures during surgical implantation, or to enable the instrument 10 to pass through small lumen vessels during intra-vascular implantation. The body 20, for example, can have a cylindrical portion with a diameter from 0.5-4 mm.

FIG. 2 is a cross-sectional view showing a portion of the instrument 10 of FIG. 1. Referring to FIGS. 1 and 2 together, the electrode contacts 30a-b are conductive bands extending around at least a portion of the body 20. A first electrode contact 30a is coupled to a first conductive line 32a, and a second electrode contact 30b is coupled to a second conductive line 32b. The first and second conductive lines 32a-b extend along the body 20 to a terminal 34 at the proximal end 22 of the body 20 (see FIG. 1). The electrode contacts 30a-b can be composed of titanium or other suitably conductive materials to either sense electrical activity from an adjacent neural structure or deliver electrical stimulation to the adjacent neural structure. For example, the first electrode contact 30a can be a sensor and the second electrode contact 30b can provide stimulation. Alternatively, the electrode contacts 30a-b can time multiplex sensing and stimulation functions such that one or both of the electrode contacts senses electrical activity during a sensing phase and then delivers electrical stimulation during a stimulation phase.

The electrode contacts 30a-b can be biased at different polarities for producing a bipolar field at the stimulation site, or they can be biased at the same polarity to produce a unipolar portion of a field at the stimulation site. In the case of biasing both of the electrode contacts 30a-b at the same polarity, a third electrode is generally positioned at a different location along the body 20 or attached to the patient at a different location to establish the electrical field. The embodiment of the instrument 10 shown in FIGS. 1 and 2 includes two electrode contacts 30a and 30b, but the instrument 10 can have any number of electrode contacts including a single contact or more than two contacts. Several deep brain electrodes, for example, have three electrode contacts.

The embodiment of the instrument 10 illustrated in FIG. 1 includes three markers 40a-c embedded in or otherwise carried by the body 20. The makers 40a-c are arranged along a common axis of the body 20 in the embodiment of the instrument 10 shown in FIG. 1, but they can be arranged in other configurations as explained below. The instrument 10 can have fewer markers, such as a single marker, or more markers depending upon the particular application. When the instrument 10 has two markers on a common axis, such as markers 40a and 40c, the x-y-z coordinate of the electrode contacts 30a-b and the angular orientation of the body 20 along two axes can be determined.

Referring to FIG. 2, the marker 40c includes a transponder 42 including a core 44, a coil 46 around the core 44, and a capacitor 48 electrically coupled to the coil 46. The core 44 is typically composed of ferrite, and the coil 46 includes a plurality of windings of a wire around the core. The transponder 42 can be contained in a capsule 49 within the body 20. In other embodiments, the transponder 42 is molded into the body 20 such that it does not include a separate capsule. The transponder 42 in FIG. 2 has a cross-sectional dimension less than that of the body 20. For example, the transponder 42 can have a cylindrical portion with a diameter from 0.5-3 mm, and desirably from 1-2 mm. The transponder 42 is a resonating magnetic circuit that receives a wirelessly transmitted excitation energy and produces a wirelessly transmitted location signal in response to the excitation energy. The transponder 42 accordingly has a resonant frequency at which the excitation energy powers the transponder.

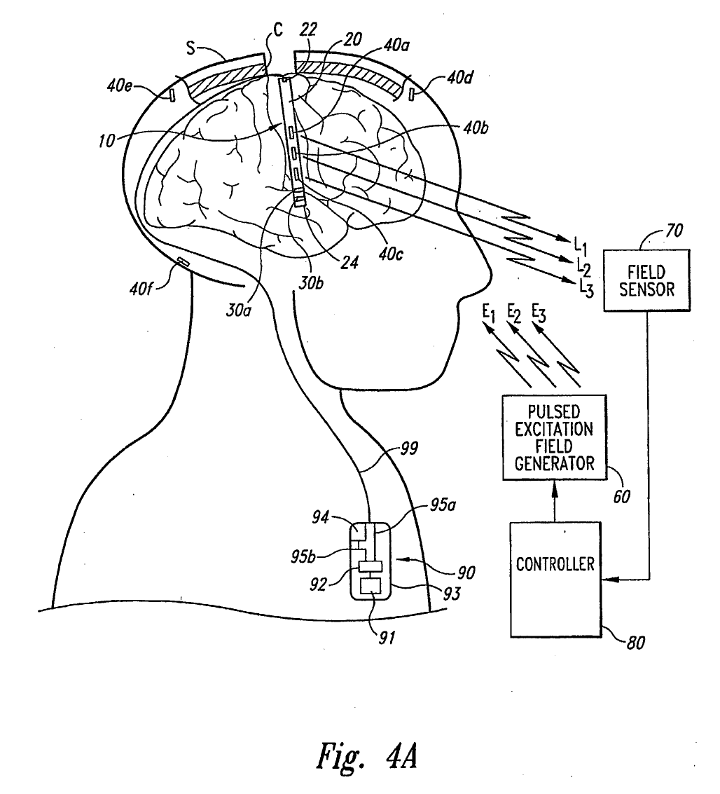

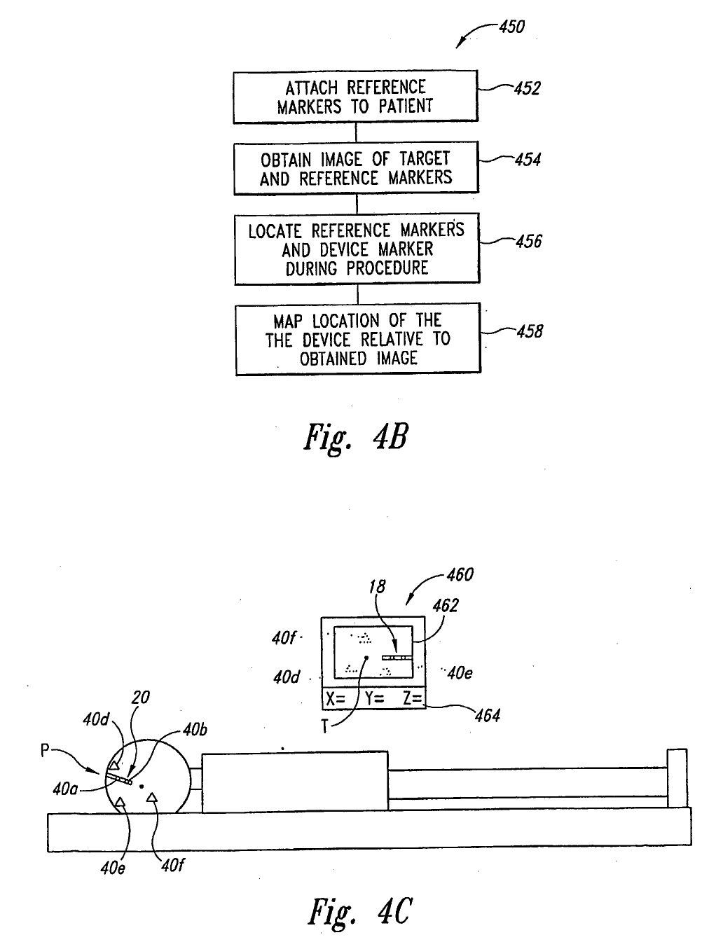

Referring to FIGS. 1 and 2 together, the markers 40a and 40b can also have transponders 42 (FIG. 2) as described above with reference to marker 40c. Each of the markers 40a-c can have transponders with unique resonant frequencies such that the first marker 40a has a first transponder with a first resonant frequency, the second marker 40b has a second transponder with a second frequency, and the third marker 40c has a third transponder with a third resonant frequency. As explained in more detail with reference to FIG. 4, the transponders 42 are typically energized by a pulsed magnetic excitation field that is time and frequency mutliplexed to independently energize each transponder.

FIG. 3 is an elevational view of another embodiment of the instrument 10 in accordance with the invention. In this embodiment, the markers 40a-c are located on different axes of the body 20 so that the angular orientation relative to the longitudinal axis of the body 20 (i.e., the “roll”) can also be determined. The instrument 10 further includes a drug delivery element 50 for delivering chemicals to the target neural structure. The drug delivery element 50 can be an opening through which a drug can be injected into the patient, or the drug delivery element can be a suitable material that elutes the drug over a period of time. The embodiment of the instrument 10 illustrated in FIG. 3 is shown as having both the electrode contacts 30 and the drug delivery element 50, but in other embodiments the instrument 10 can include just the drug delivery element 50 without the electrode contacts 30.

### C. Systems and Procedures of Using Implantable Instruments

FIG. 4A is a schematic view illustrating an embodiment of a localization system and an embodiment of an implantable stimulation system for use with any of the embodiments of the instrument 10 illustrated and/or described above with reference to FIGS. 1-3. The localization system and the markers 40a-c are used to determine the location of the instrument 10 during and after implantation. The stimulation system and the electrode contacts 30a-b are used to sense and/or stimulate a target neural structure within the patient. The stimulation system can also operate a drug delivery element as described above with reference to FIG. 3.

The localization system includes an excitation source 60 (e.g., a pulsed magnetic field generator), a sensor assembly 70, and a controller 80 coupled to both the excitation source 60 and the sensor assembly 70. The excitation source 60 generates an excitation energy to energize at least one of the markers 40a-c on the instrument 10. The embodiment of the excitation source 60 shown in FIG. 4 produces a pulsed magnetic field at different frequencies. For example, the excitation source 60 can frequency multiplex the magnetic field at a first frequency E₁ to energize the first marker 40a, a second frequency E₂ to energize the second marker 40b, and a third frequency E₃ to energize the third marker 40c. In response to the excitation energy, the markers 40a-c generate location signals L₁₋₃ at unique response frequencies. More specifically, the first marker 40a generates a first location signal L₁ at a first frequency in response to the excitation energy at the first frequency E₁, the second marker 40b generates a second location signal L₂ at a second frequency in response to the excitation energy at the second frequency E₂, and the third marker 40c generates a third location signal L₃ at a third frequency in response to the excitation energy at the third frequency E₃.

The sensor assembly 70 can include a plurality of coils to sense the location signals L₁₋₃ from the markers 40a-c. The sensor assembly 70 can be a flat panel having a plurality of coils that are at least substantially coplanar relative to each other. In other embodiments, the sensor assembly 70 may be a non-planar array of coils.

The controller 80 includes hardware, software or other computer-operable media containing instructions that operate the excitation source 60 to multiplex the excitation energy at the different frequencies E₁₋₃. For example, the controller 80 causes the excitation source 60 to generate the excitation energy at the first frequency E₁ for a first excitation period, and then the controller 80 causes the excitation source 60 to terminate the excitation energy at the first frequency E₁ for a first sensing phase during which the sensor assembly 70 senses the first location signal L₁ from the first marker 40a without the presence of the excitation energy at the first frequency E₁. The controller 80 then causes the excitation source 60 to: (a) generate the second excitation energy at the second frequency E₂ for a second excitation period; and (b) terminate the excitation energy at the second frequency E₂ for a second sensing phase during which the sensor assembly 70 senses the second location signal L₂ from the second marker 40b without the presence of the second excitation energy at the second frequency E₂. The controller 80 then repeats this operation with the third excitation energy at the third frequency E₃ such that the third marker 40c transmits the third location signal L₃ to the sensor assembly 70 during a third sensing phase. As such, the excitation source 60 wirelessly transmits the excitation energy in the form of pulsed magnetic fields at the resonant frequencies of the markers 40a-c during excitation periods, and the markers 40a-c wirelessly transmit the location signals L₁₋₃ to the sensor assembly 70 during sensing phases.

The computer-operable media in the controller 80, or in a separate signal processor, also includes instructions to determine the absolute positions of each of the markers 40a-c in a three-dimensional reference frame. Based on signals provided by the sensor assembly 70 that correspond to the magnitude of each of the location signals L₁₋₃, the controller 80 and/or a separate signal processor calculates the absolute coordinates of each of the markers 40a-c in the three-dimensional reference frame.

One procedure for implanting the instrument 10 into the patient includes attaching reference markers 40d-f to the patient and acquiring reference images showing the position of the reference markers 40d-f relative to the target neural structure using MRI images, CT images, radiographic images, or other suitable types of images. The reference markers 40d-f can be adhered to the patient using an external patch or anchored to the patient's skull. The instrument 10 is then implanted into the patient by moving the distal end 24 of the body 20 into the brain along a selected trajectory. As the instrument 10 is inserted into the patient, the markers 40a-f are individually energized by the excitation source 60 at six different frequencies, and the sensor assembly 70 receives independent location signals from each of the markers 40a-f. The controller 80 and/or a separate signal processor then calculates the absolute position of each marker in a three-dimensional reference frame. The controller 80 can also calculate: (a) the location of the electrode contacts 30a-b using the absolute locations of the markers 40a-c; and (b) the location of the target neural structure using the absolute locations of the markers 40d-f. Based on the calculated locations of the electrode contacts 30a-b and the target neural structure, the controller 80 can further calculate the relative offset between the electrode contacts 30a-b and the target neural structure in real time.

The instrument 10 and localization system enable a practitioner to track the location of the instrument 10 relative to the target neural structure as it is being implanted into the patient and at any time after implantation. The location system illustrated in FIG. 4 can calculate the absolute position of each individual marker 40 at a frequency of approximately 1 ms to 1.0 second. Additionally, the location system can provide the absolute locations of the markers 40, the electrode contacts 30a-b, and/or the target neural structure either individually or relative to one another within a latency of 10 ms to 2 seconds from the time the localization signals were transmitted from the markers 40. The location system accordingly provides real-time tracking to an operator to ensure that the electrode contacts 30a-b are positioned at a desired stimulation site relative to the target neural structure. This is expected to enhance the efficacy of the stimulation and mitigate collateral stimulation of neighboring neural structures because the electrode contacts will be located where the electrical field can produce the desired results at the low stimulation levels. The location system also enhances the ability to insert the instrument 10 along a path that mitigates damage to collateral neural structures or other tissue. This is expected to increase the reliability and safety of implanting electrodes and sensors into deep brain regions.

FIG. 4A also illustrates an embodiment of a stimulation system for use with the instrument 10. In this embodiment, the stimulation system can include the instrument 10, an implantable stimulus unit 90 configured to be implanted in the patient, and a flexible lead 99 coupled to the instrument 10 and the stimulus unit 90. As shown in FIGS. 1 and 3, for example, the flexible lead 99 is connected to the terminal 34 at the proximal end 22 of the body 20. Referring back to FIG. 4, the stimulus unit 90 can include a controller 91, a pulse generator 92 operatively coupled to the controller to generate a stimulation pulse, and a housing 93 containing the controller 91 and the pulse generator 92. The housing 93 can be a dielectric material, or it can be a conductive material with a dielectric coating. The housing 93 can have an electrode contact 94 to provide an additional electrode contact for forming an electrical field between the electrode contacts 30a-b and the electrode contact 94. The electrode contact 94 can be an electrically conductive portion of the housing 93 or a separate electrically conductive member attached to the housing 93. The stimulus unit 90 can further include conductive lines 95a-b. A first conductive line 95a can include one or more conductive elements that are connected to the lead 99, and a second conductive line 95b can be coupled to the electrode contact 94.

In operation, the controller 91 causes the pulse generator 92 to generate an electrical pulse that is sent along the first conductive line 95a and through the lead 99 to the electrode contacts 30a-b. The controller 90 can optionally cause the pulse generator 92 to bias the electrode contact 94 in addition to the electrode contacts 30a-b. Several suitable stimulation parameters are described in the art for treating epilepsy, movement disorders, and other neurological diseases and/or disorders using deep brain stimulation of the thalamus, the vagas nerve, and/or other deep brain neural structures.

FIG. 4B is a flow chart illustrating a method 450 of using an instrument in accordance with an embodiment of the invention, and FIG. 4C is a schematic view illustrating aspects of an embodiment of the method 450. The method 450 for performing a diagnostic or therapeutic procedure on a patient includes a first stage 452 in which reference markers 40d-f (FIG. 4C) are attached to a patient P. The method 450 further includes a second stage 454 that includes obtaining one or more reference images showing the position of the reference markers 40d-f relative to the target T using MRI images, CT images, radiographic images; ultrasonic images, or other suitable types of images as explained above with reference to FIG. 4A. The reference markers 40d-f can be adhered to the patient using an external patch, implanted in tissue, or otherwise anchored to the bone structure of the patient. One aspect of the first and second stages 452 and 454 is that the markers 40d-f can be attached to the patient during a diagnostic stage of treating the patient before obtaining the reference images. The markers can then be left in the patient for a long period of time because they are not hard-wired to any external excitation or sensing devices. The patient can accordingly be moved for further diagnostic procedures or therapeutic procedures at a later time or in a different location.

The method 450 continues with a third stage 456 in which the reference markers 40d-f and device markers 40a-b (FIG. 4C) are located during a diagnostic and/or therapeutic procedure. The reference markers 40d-f and device markers 40a-b shown in FIG. 4C can be located simultaneously, or at least substantially simultaneously, in real time during the procedure as described above with reference to FIG. 4A. The method 450 further includes a fourth stage 458 in which the location of the device 20 is mapped or otherwise presented relative to the target T by superimposing a representation of the instrument on a display of a reference image that was previously obtained.

The fourth stage 458 of the method 450 can have several different embodiments. Referring to FIG. 4C, for example, the system can further include a display 460 that provides an image 462 that has been registered to the proper orientation using the reference images that were obtained in the earlier stages of the method and a reference frame defined by the reference markers 40d-f (shown in phantom in the image 462). The position of the distal section 18 of the instrument can be mapped (e.g., superimposed) onto the registered image 462 to illustrate the relative orientation between the instrument and the target T. The display 460 can further include alphanumeric indicators 464 illustrating the relative displacement between the distal section 18 of the instrument and the target T. U.S. Pat. Nos. 5,729,129 and 6,161,032, which are herein incorporated by reference, disclose processes for displaying the position of the device 20 on the display 460 by superimposing a representation of the device on previously acquired images of the patient.

The systems and methods set forth above with respect to FIGS. 4A-4C that use wireless markers provide several advantages over conventional systems using wired transponders. For example, U.S. Pat. Nos. 5,729,129 and 6,161,032 disclose “wired” systems in which magnetic field sensors attached to the patient or a probe are hard-wired to a receiver to detect the position and orientation of medical probes within the body of a patient. U.S. Pat. No. 6,161,032 discloses a system having a wired field transmitter attached to the end of a probe (e.g., a catheter), three wired reference assemblies that can be attached to the patient, and a calibration array that is separate from the patient. In a typical application, it appears that a patient initially undergoes a diagnostic procedure in which the target (e.g., a soft tissue lesion) is imaged. The patient then proceeds to a therapeutic procedure at a later point in time during which the wired reference assemblies are attached to desired locations on the patient. The system is then calibrated with the patient in position for the therapy by locating the wired reference assemblies using either a calibration array or a probe that is manually placed on the reference assemblies. The image of the patient is then registered with respect to the external reference frame defined by the three reference assemblies. At this point, the patient is then ready to actually undergo the therapeutic procedure in which the probe is located relative to the reference transducers. The position of the probe is then mapped to the image to provide the practitioner a visual representation of the relative position between the probe and the target.

One problem with such wired systems is that the reference assemblies are attached to the patient after obtaining the diagnostic images. The system is thus manually calibrated before performing the therapeutic procedure. This is a relatively time consuming aspect of the procedure that reduces the utilization of expensive equipment and facilities associated with surgical or therapeutic procedures. Another problem with such systems is that the reference assemblies may not be accurately positioned relative to the target such that the external reference frame defined by the reference assemblies introduces systemic errors that decrease the accuracy of the measurements. Therefore, wired magnetic tracking systems are not expected to provide satisfactory results for many applications.

In contrast to the wired systems, the systems and methods set forth in FIGS. 4A-4C that use wireless markers increase the utilization of expensive facilities and accurately localize the instrument. The reference markers 40d-f of the system illustrated in FIG. 4C are accurately imaged and localized during an initial diagnostic stage of a therapy. This eliminates having to calibrate the system and determine the reference frame while a patient is positioned at a treatment site immediately before a treatment as required in U.S. Pat. No. 6,161,032. As a result, the inventive systems and methods increase the utilization of expensive operating rooms or other equipment. The inventive systems and methods also reduce systemic errors caused by inaccurately positioning reference assemblies on the patient or inaccurately placing a probe tip on a reference assembly as disclosed in U.S. Pat. No. 6,161,032.

The systems and methods described above with reference to FIGS. 4B and 4C also provide more accurate measurements because the reference markers 40d-f inherently move with the patient to enhance the accuracy with which the instrument is positioned relative to the target. For example, the reference markers 40d-f can be implanted very close to soft tissue targets or dynamic organs (e.g., the heart or lungs) so that the reference frame defined by the markers moves with the target. Additionally, because the reference markers and the device markers are located concurrently during a procedure, the dynamic measurement of the reference frame automatically compensates for patient movement. This eliminates having to calibrate the reference frame defined by the markers and having to re-register or re-map images relative to the markers. As a result, the systems and methods described above with reference to FIGS. 4B and 4C provide greater accuracy and enable faster processing times for diagnostic and/or therapeutic procedures.

The systems set forth in FIGS. 4B and 4C further provide additional comfort to the patient throughout the diagnostic and therapeutic procedures. Because the reference markers 40d-f are wireless, they can remain in the patient after implantation for an indefinite period of time without having any leads or markers external to the patient. This allows the patient to go about normal daily functions without complications caused by external lead wires, which is particularly beneficial for treatments that involve one or more procedures over a number of days or weeks. The markers 40d-f, moreover, do not generate a significant amount of heat and they are relatively small. Thus, they do not cause uncomfortable sensations or pain.

### D. Specific Embodiments of Markers and Localization Systems

The following specific embodiments of markers, excitation sources, sensors and controllers 80 provide additional details to implement the systems and processes described above with reference to FIGS. 1-4C. The present inventors overcame many challenges to develop markers and localization systems that accurately determine the location of a marker which (a) produces a wirelessly transmitted location signal in response to a wirelessly transmitted excitation energy, and (b) has a cross-section small enough to be implanted in the brain of the patient. The following specific embodiments are described in sufficient detail to enable a person skilled in the art to make and use such a localization system for implanting a deep brain electrode, but the invention is not limited to the following embodiments of markers, excitation sources, sensor assemblies and/or controllers.

1. Markers

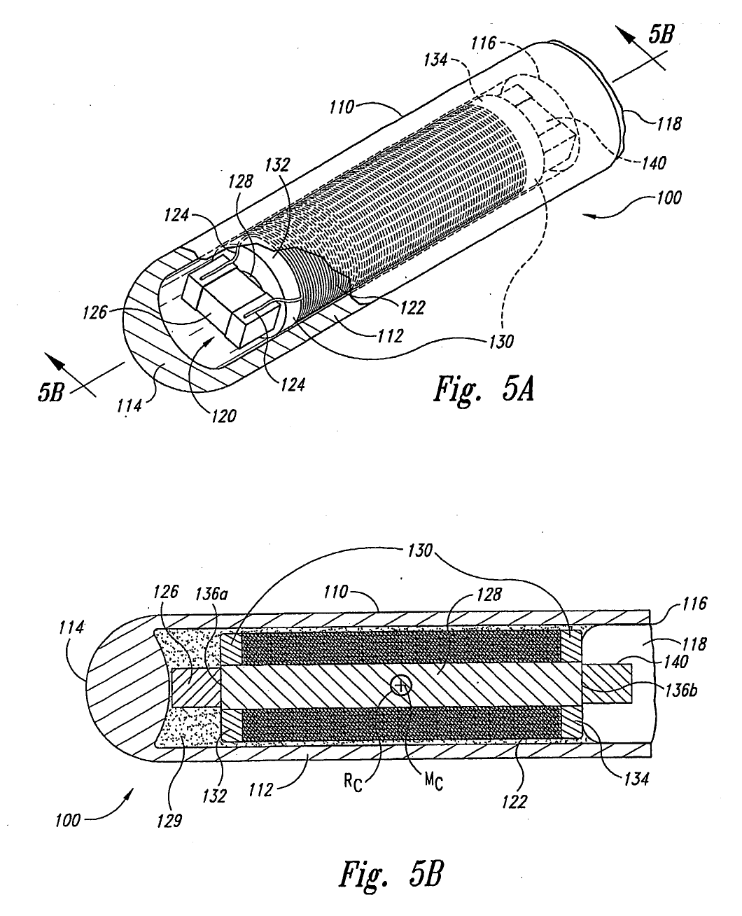

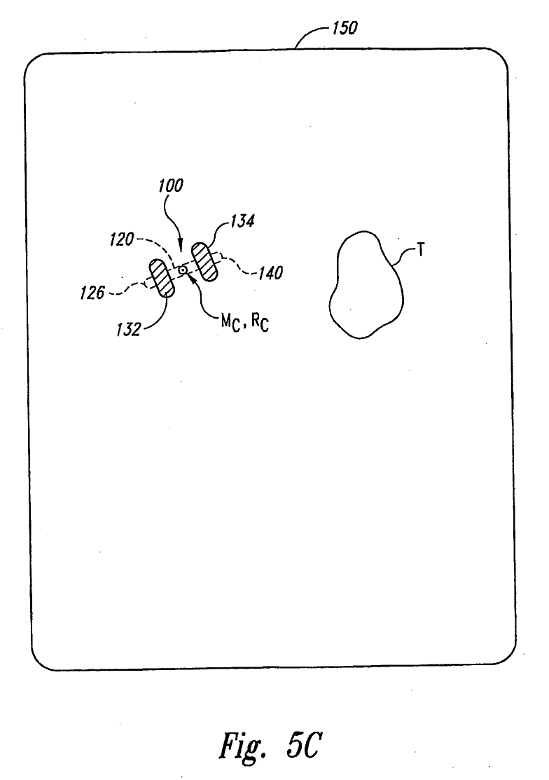

FIG. 5A is an isometric view of a marker 100 for use with the instrument 10 (FIGS. 1-3). The embodiment of the marker 100 shown in FIG. 5A includes a casing 110 and a magnetic transponder 120 (e.g., a resonating circuit) in the casing 110. The casing 110 is a barrier configured to be implanted in the patient, or encased within the body 20 (FIG. 1) of the instrument 10. The casing 110 can alternatively be configured to be adhered externally to the body 20 or the skin of the patient, or otherwise attached to the body 20 or the patient. The casing 110 can be a generally cylindrical capsule that is sized to fit within the body 20 (FIG. 1), but the casing 110 can have other configurations and be larger or smaller. The casing 110, for example, can have barbs or other features to anchor the casing 110 in soft tissue or an adhesive for attaching the casing 110 externally to the skin of a patient. Suitable anchoring mechanisms for securing the marker 100 to a patient are disclosed in International Publication No. WO 02/39917 A1, which designates the United States and is incorporated herein by reference. In one embodiment, the casing 110 includes (a) a capsule or shell 112 having a closed end 114 and an open end 116, and (b) a sealant 118 in the open end 116 of the shell 112. The casing 110 and the sealant 118 can be made from plastics, ceramics, glass or other suitable biocompatible materials.

The magnetic transponder 120 can include a resonating circuit that wirelessly transmits a location signal in response to a wirelessly transmitted excitation field as described above. In this embodiment, the magnetic transponder 120 comprises a coil 122 defined by a plurality of windings of a conductor 124. Many embodiments of the magnetic transponder 120 also include a capacitor 126 coupled to the coil 122. The coil 122 resonates at a selected resonant frequency. The coil 122 can resonate at a resonant frequency solely using the parasitic capacitance of the windings without having a capacitor, or the resonant frequency can be produced using the combination of the coil 122 and the capacitor 126. The coil 122 accordingly generates an alternating magnetic field at the selected resonant frequency in response to the excitation energy either by itself or in combination with the capacitor 126. The conductor 124 of the illustrated embodiment can be hot air or alcohol bonded wire having a gauge of approximately 45-52. The coil 122 can have 800-1000 turns, and the windings are preferably wound in a tightly layered coil. The magnetic transponder 120 can further include a core 128 composed of a material having a suitable magnetic permeability. For example, the core 128 can be a ferromagnetic element composed of ferrite or another material. The magnetic transponder 120 can be secured to the casing 110 by an adhesive 129.

The marker 100 also includes an imaging element that enhances the radiographic image of the marker to make the marker more discernible in radiographic images. The imaging element also has a radiographic profile in a radiographic image such that the marker has a radiographic centroid at least approximately coincident with the magnetic centroid of the magnetic transponder 120. As explained in more detail below, the radiographic and magnetic centroids do not need to be exactly coincident with each other, but rather can be within an acceptable range.

FIG. 5B is a cross-sectional view of the marker 100 along line 5B-5B of FIG. 5A that illustrates an imaging element 130 in accordance with an embodiment of the invention. The imaging element 130 illustrated in FIGS. 5A-B includes a first contrast element 132 and second contrast element 134. The first and second contrast elements 132 and 134 are generally configured with respect to the magnetic transponder 120 so that the marker 100 has a radiographic centroid R_{c} that is at least substantially coincident with the magnetic centroid M_{c} of the magnetic transponder 120. For example, when the imaging element 130 includes two contrast elements, the contrast elements can be arranged symmetrically with respect to the magnetic transponder 120 and/or each other. The contrast elements can also be radiographically distinct from the magnetic transponder 120. In such an embodiment, the symmetrical arrangement of distinct contrast elements enhances the ability to accurately determine the radiographic centroid of the marker 100 in a radiographic image.

The first and second contrast elements 132 and 134 illustrated in FIGS. 5A-B are continuous rings positioned at opposing ends of the core 128. The first contrast element 132 can be at or around a first end 136a of the core 128, and the second contrast element 134 can be at or around a second end 136b of the core 128. The continuous rings shown in FIGS. 5A-B have substantially the same diameter and thickness. The first and second contrast elements 132 and 134, however, can have other configurations and/or be in other locations relative to the core 128 in other embodiments. For example, the first and second contrast elements 132 and 134 can be rings with different diameters and/or thicknesses.

The radiographic centroid of the image produced by the imaging element 130 does not need to be absolutely coincident with the magnetic centroid M_{c}, but rather the radiographic centroid and the magnetic centroid should be within an acceptable range. For example, the radiographic centroid R_{c} can be considered to be at least approximately coincident with the magnetic centroid M_{c} when the offset between the centroids is less than approximately 5 mm. In more stringent applications, the magnetic centroid M_{c} and the radiographic centroid R_{c} are considered to be at least substantially coincident with each other when the offset between the centroids is 2 mm or less. In other applications, the magnetic centroid M_{c} is at least approximately coincident with the radiographic centroid R_{c} when the centroids are spaced apart by a distance not greater than half the length of the magnetic transponder 120 and/or the marker 100.

The imaging element 130 can be made from a material and configured appropriately to absorb a high fraction of incident photons of a radiation beam used for producing the radiographic image. For example, when the imaging radiation has high acceleration voltages in the megavoltage range, the imaging element 130 is made from, at least in part, high density materials with sufficient thickness and cross-sectional area to absorb enough of the photon fluence incident on the imaging element to be visible in the resulting radiograph. Many high energy beams used for therapy have acceleration voltages of 6 MV-25 MV, and these beams are often used to produce radiographic images in the 5 MV-10 MV range, or more specifically in the 6 MV-8 MV range. As such, the imaging element 130 can be made from a material that is sufficiently absorbent of incident photon fluence to be visible in an image produced using a beam with an acceleration voltage of 5 MV-10 MV, or more specifically an acceleration voltage of 6 MV-8 MV.

Several specific embodiments of imaging elements 130 can be made from gold, tungsten, platinum and/or other high density metals. In these embodiments the imaging element 130 can be composed of materials having a density of 19.25 g/cm³ (density of tungsten) and/or a density of approximately 21.4 g/cm³ (density of platinum). Many embodiments of the imaging element 130 accordingly have a density not less than 19 g/cm³. In other embodiments, however, the material(s) of the imaging element 130 can have a substantially lower density. For example, imaging elements with lower density materials are suitable for applications that use lower energy radiation to produce radiographic images. Moreover, the first and second contrast elements 132 and 134 can be composed of different materials such that the first contrast element 132 can be made from a first material and the second contrast element 134 can be made from a second material.

Referring to FIG. 5B, the marker 100 can further include a module 140 at an opposite end of the core 128 from the capacitor 126. In the embodiment of the marker 100 shown in FIG. 5B, the module 140 is configured to be symmetrical with respect to the capacitor 126 to enhance the symmetry of the radiographic image. As with the first and second contrast elements 132 and 134, the module 140 and the capacitor 126 are arranged such that the magnetic centroid of the marker is at least approximately coincident with the radiographic centroid of the marker 100. The module 140 can be another capacitor that is identical to the capacitor 126, or the module 140 can be an electrically inactive element. Suitable electrically inactive modules include ceramic blocks shaped like the capacitor 126 and located with respect to the coil 122, the core 128 and the imaging element 130 to be symmetrical with each other. In still other embodiments the module 140 can be a different type of electrically active element electrically coupled to the magnetic transponder 120.

One specific process of using the marker involves imaging the marker using a first modality and then tracking the target of the patient and/or the marker using a second modality. For example, the location of the marker relative to the target can be determined by imaging the marker and the target using radiation. The marker and/or the target can then be localized and tracked using the magnetic field generated by the marker in response to an excitation energy.

The marker 100 shown in FIGS. 5A-B is expected to provide an enhanced radiographic image compared to conventional magnetic markers for more accurately determining the relative position between the marker and the target of a patient. FIG. 5C, for example, illustrates a radiographic image 150 of the marker 100 and a target T of the patient. The first and second contrast elements 132 and 134 are expected to be more distinct in the radiographic image 150 because they can be composed of higher density materials than the components of the magnetic transponder 120. The first and second contrast elements 132 and 134 can accordingly appear as bulbous ends of a dumbbell shape in applications in which the components of the magnetic transponder 120 are visible in the image. In certain megavolt applications, the components of the magnetic transponder 120 may not appear at all on the radiographic image 150 such that the first and second contrast elements 132 and 134 will appear as distinct regions that are separate from each other. In either embodiment, the first and second contrast elements 132 and 134 provide a reference frame in which the radiographic centroid R_{c} of the marker 100 can be located in the image 150. Moreover, because the imaging element 130 is configured so that the radiographic centroid R_{c} is at least approximately coincident with the magnetic centroid M_{c}, the relative offset or position between the target T and the magnetic centroid M_{c} can be accurately determined using the marker 100. The embodiment of the marker 100 illustrated in FIGS. 5A-C, therefore, is expected to mitigate errors caused by incorrectly estimating the radiographic and magnetic centroids of markers in radiographic images.

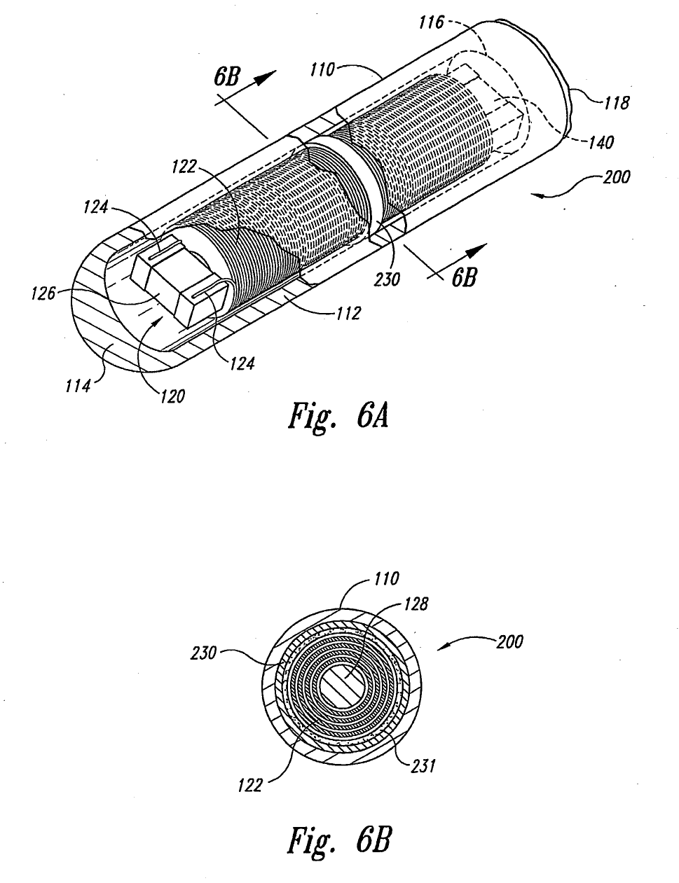

FIG. 6A is an isometric view of a marker 200 with a cut-away portion to illustrate internal components, and FIG. 6B is a cross-sectional view of the marker 200 taken along line 6B-6B of FIG. 6A. The marker 200 is similar to the marker 100 shown above in FIG. 5A, and thus like reference numbers refer to like components. The marker 200 differs from the marker 100 in that the marker 200 includes an imaging element 230 defined by a single contrast element. The imaging element 230 is generally configured relative to the magnetic transponder 120 so that the radiographic centroid of the marker 200 is at least approximately coincident with the magnetic centroid of the magnetic transponder 120. The imaging element 230, more specifically, is a ring extending around the coil 122 at a medial region of the magnetic transponder 120. The imaging element 230 can be composed of the same materials described above with respect to the imaging element 130 in FIGS. 5A-B. The imaging element 230 can have an inner diameter that is approximately equal to the outer diameter of the coil 122, and an outer diameter within the casing 110. As shown in FIG. 6B, however, a spacer 231 can be between the inner diameter of the imaging element 230 and the outer diameter of the coil 122.

The marker 200 is expected to operate in a manner similar to the marker 100 described above. The marker 200, however, does not have two separate contrast elements that provide two distinct, separate points in a radiographic image. The imaging element 230 is still highly useful in that it identifies the radiographic centroid of the marker 200 in a radiographic image, and it can be configured so that the radiographic centroid of the marker 200 is at least approximately coincident with the magnetic centroid of the magnetic transponder 120.

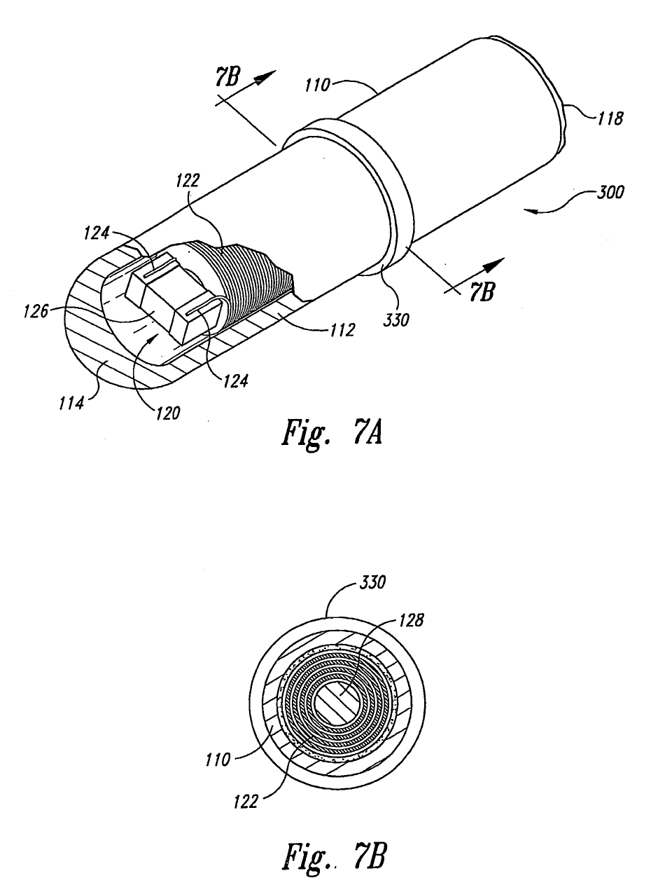

FIG. 7A is an isometric view of a marker 300 having a cut-away portion, and FIG. 7B is a cross-sectional view of the marker 300 taken along line 7B-7B of FIG. 7A. The marker 300 is substantially similar to the marker 200 shown in FIGS. 6A-B, and thus like reference numbers refer to like components in FIGS. 5A-7B. The imaging element 330 can be a high density ring configured relative to the magnetic transponder 120 so that the radiographic centroid of the marker 300 is at least approximately coincident with the magnetic centroid of the magnetic transponder 120. The marker 300, more specifically, includes an imaging element 330 around the casing 110. The marker 300 is expected to operate in much the same manner as the marker 200 shown in FIGS. 6A-B.

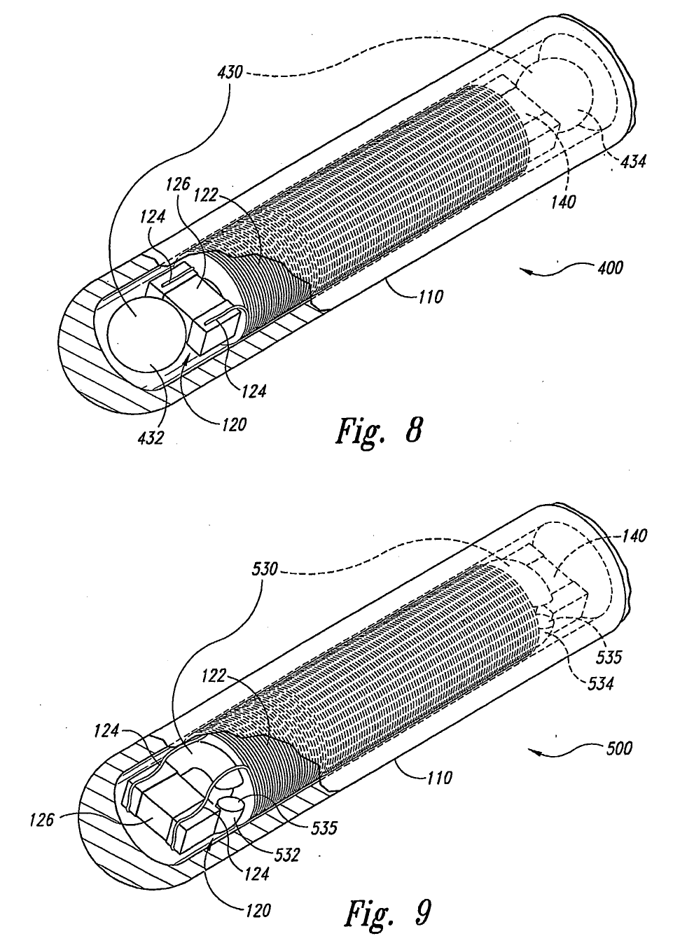

FIG. 8 is an isometric view with a cut-away portion illustrating a marker 400 in accordance with another embodiment of the invention. The marker 400 is similar to the marker 100 shown in FIGS. 5A-C, and thus like reference numbers refer to like components in these Figures. The marker 400 has an imaging element 430 including a first contrast element 432 at one end of the magnetic transponder 120 and a second contrast element 434 at another end of the magnetic transponder 120. The first and second contrast elements 432 and 434 are spheres composed of suitable high density materials. The contrast elements 432 and 434, for example, can be composed of gold, tungsten, platinum or other suitable high-density materials for use in radiographic imaging. The marker 400 is expected to operate in a manner similar to the marker 100, as described above.

FIG. 9 is an isometric view with a cut-away portion of a marker 500 in accordance with yet another embodiment of the invention. The marker 500 is substantially similar to the markers 100 and 400 shown in FIGS. 5A and 8, and thus like reference numbers refer to like components in these Figures. The marker 500 includes an imaging element 530 including a first contrast element 532 and a second contrast element 534. The first and second contrast elements 532 and 534 can be positioned proximate to opposing ends of the magnetic transponder 120. The first and second contrast elements 532 and 534 can be discontinuous rings having a gap 535 to mitigate eddy currents. The contrast elements 532 and 534 can be composed of the same materials as described above with respect to the contrast elements of other imaging elements in accordance with other embodiments of the invention.

Additional embodiments of markers in accordance with the invention can include imaging elements incorporated into or otherwise integrated with the casing 110, the core 128 (FIG. 5B) of the magnetic transponder 120, and/or the adhesive 129 (FIG. 5B) in the casing. For example, particles of a high density material can be mixed with ferrite and extruded to form the core 128. Alternative embodiments can mix particles of a high density material with glass or another material to form the casing 110, or coat the casing 110 with a high-density material. In still other embodiments, a high density material can be mixed with the adhesive 129 and injected into the casing 110. Any of these embodiments can incorporate the high density material into a combination of the casing 110, the core 128 and/or the adhesive 129. Suitable high density materials can include tungsten, gold and/or platinum as described above.

The markers described above with reference to FIGS. 5A-9 can be used for the markers 40 in the instrument 10 (FIGS. 1-4). The instrument 10 can have several markers with the same type of imaging elements, or markers with different imaging elements can be used with the same instrument. Several additional details of these markers and other embodiments of markers are described in U.S. application Ser. Nos. 10/334,698 and 10/746,888, which are incorporated herein by reference. For example, the markers may not have any imaging elements for applications with lower energy radiation, or the markers may have reduced volumes of ferrite and metals to mitigate issues with MRI imaging as set forth in U.S. application Ser. No. 10/334,698.

2. Localization Systems

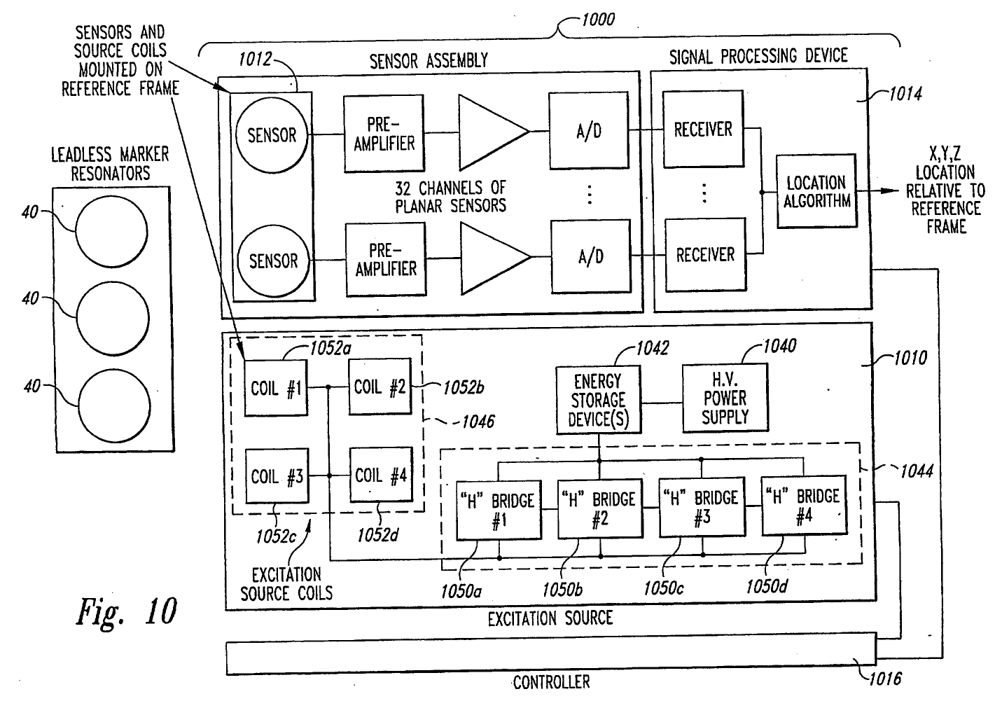

FIG. 10 is a schematic block diagram of a localization system 1000 for determining the absolute location of the markers 40 (shown schematically) relative to a reference frame. The localization system 1000 includes an excitation source 1010, a sensor assembly 1012, a signal processor 1014 operatively coupled to the sensor assembly 1012, and a controller 1016 operatively coupled to the excitation source 1010 and the signal processor 1014. The excitation source 1010 is one embodiment of the excitation source 60 described above with reference to FIG. 4; the sensor assembly 1012 is one embodiment of the sensor assembly 70 described above with reference to FIG. 4; and the controller 1016 is one embodiment of the controller 80 described above with reference to FIG. 4.

The excitation source 1010 is adjustable to generate a magnetic field having a waveform with energy at selected frequencies to match the resonant frequencies of the markers 40. The magnetic field generated by the excitation source 1010 energizes the markers at their respective frequencies. After the markers 40 have been energized, the excitation source 1010 is momentarily switched to an “off” position so that the pulsed magnetic excitation field is terminated while the markers wirelessly transmit the location signals. This allows the sensor assembly 1012 to sense the location signals from the markers 40 without measurable interference from the significantly more powerful magnetic field from the excitation source 1010. The excitation source 1010 accordingly allows the sensor assembly 1012 to measure the location signals from the markers 40 at a sufficient signal-to-noise ratio so that the signal processor 1014 or the controller 1016 can accurately calculate the absolute location of the markers 40 relative to a reference frame.

a. Excitation Sources

Referring still to FIG. 10, the excitation source 1010 includes a high voltage power supply 1040, an energy storage device 1042 coupled to the power supply 1040, and a switching network 1044 coupled to the energy storage device 1042. The excitation source 1010 also includes a coil assembly 1046 coupled to the switching network 1044. In one embodiment, the power supply 1040 is a 500 volt power supply, although other power supplies with higher or lower voltages can be used. The energy storage device 1042 in one embodiment is a high voltage capacitor that can be charged and maintained at a relatively constant charge by the power supply 1040. The energy storage device 1042 alternately provides energy to and receives energy from the coils in the coil assembly 1046.

The energy storage device 1042 is capable of storing adequate energy to reduce voltage drop in the energy storage device while having a low series resistance to reduce power losses. The energy storage device 1042 also has a low series inductance to more effectively drive the coil assembly 1046. Suitable capacitors for the energy storage device 1042 include aluminum electrolytic capacitors used in flash energy applications. Alternative energy storage devices can also include NiCd and lead acid batteries, as well as alternative capacitor types, such as tantalum, film, or the like.

The switching network 1044 includes individual H-bridge switches 1050 (identified individually by reference numbers 1050a-d), and the coil assembly 1046 includes individual source coils 1052 (identified individually by reference numbers 1052a-d). Each H-bridge switch 1050 controls the energy flow between the energy storage device 1042 and one of the source coils 1052. For example, H-bridge switch #1 1050a independently controls the flow of the energy to/from source coil #1 1052a, H-bridge switch #2 1050b independently controls the flow of the energy to/from source coil #2 1052b, H-bridge switch #3 1050c independently controls the flow of the energy to/from source coil #3 1052c, and H-bridge switch #4 1050d independently controls the flow of the energy to/from source coil #4 1052d. The switching network 1044 accordingly controls the phase of the magnetic field generated by each of the source coils 1052a-d independently. The H-bridges 1050 can be configured so that the electrical signals for all the source coils 1052 are in phase, or the H-bridge switches 1050 can be configured so that one or more of the source coils 1052 are 180° out of phase. Furthermore, the H-bridge switches 1050 can be configured so that the electrical signals for one or more of the source coils 1052 are between 0 and 180° out of phase to simultaneously provide magnetic fields with different phases.

The source coils 1052 can be arranged in a coplanar array that is fixed relative to the reference frame. Each source coil 1052 can be a square, planar winding arranged to form a flat, substantially rectilinear coil. The source coils 1052 can have other shapes and other configurations in different embodiments. In one embodiment, the source coils 1052 are individual conductive lines formed in a stratum of a printed circuit board, or windings of a wire in a foam frame. Alternatively, the source coils 1052 can be formed in different substrates or arranged so that two or more of the source coils are not planar with each other. Additionally, alternate embodiments of the invention may have fewer or more source coils than illustrated in FIG. 10.

The selected magnetic fields from the source coils 1052 combine to form an adjustable excitation field that can have different three-dimensional shapes to excite the markers 40 at any spatial orientation within an excitation volume. When the planar array of the source coils 1052 is generally horizontal, the excitation volume is positioned above an area approximately corresponding to the central region of the coil assembly 1046. The excitation volume is the three-dimensional space adjacent to the coil assembly 1046 in which the strength of the magnetic field is sufficient to adequately energize the markers 40.

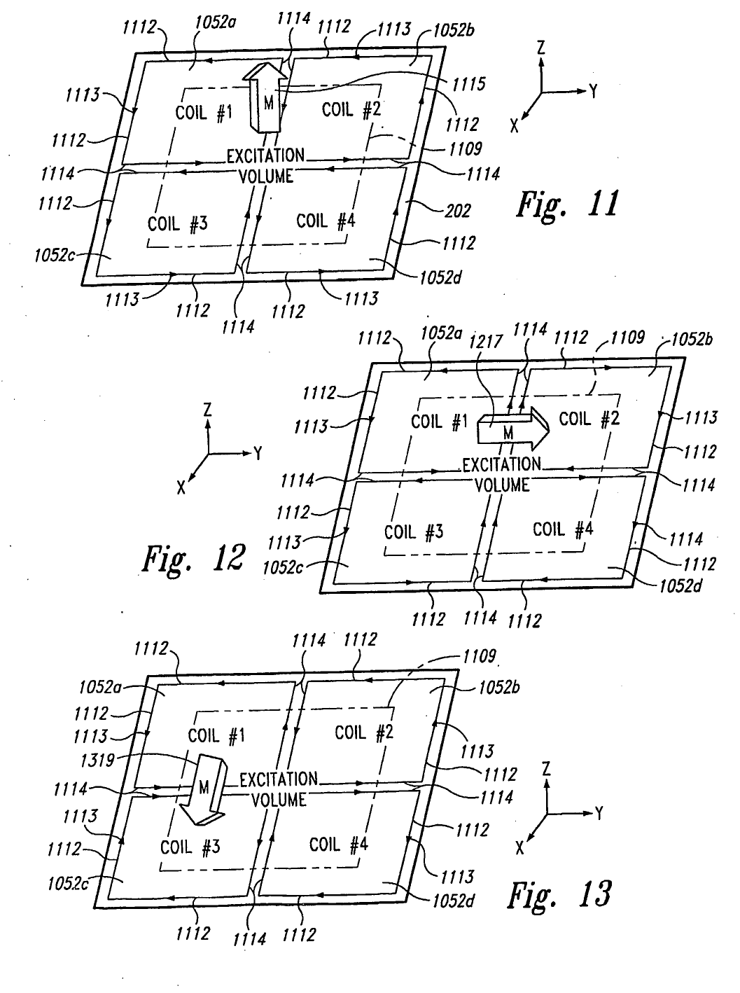

FIGS. 11-13 are schematic views of a planar array of the source coils 1052 with the alternating electrical signals provided to the source coils in different combinations of phases to generate excitation fields about different axes relative to the illustrated XYZ coordinate system. Each source coil 1052 has two outer sides 1112 and two inner sides 1114. Each inner side 1114 of one source coil 1052 is immediately adjacent to an inner side 1114 of another source coil 1052, but the outer sides 1112 of all the source coils 1052 are not adjacent to any other source coil 1052.

In the embodiment of FIG. 11, all the source coils 1052a-d simultaneously receive an alternating electrical signals in the same phase. As a result, the electrical current flows in the same direction through all the source coils 1052a-d such that a direction 1113 of the current flowing along the inner sides 1114 of one source coil (e.g., source coil 1052a) is opposite to the direction 1113 of the current flowing along the inner sides 1114 of the two adjacent source coils (e.g., source coils 1052c and 1052d). The magnetic fields generated along the inner sides 1114 accordingly cancel each other out so that the magnetic field is effectively generated from the current flowing along the outer sides 1112 of the source coils. The resulting excitation field formed by the combination of the magnetic fields from the source coils 1052a-d shown in FIG. 11 has a magnetic moment 1115 generally in the Z direction within an excitation volume 1109. This excitation field energizes markers parallel to the Z-axis or markers positioned with an angular component along the Z-axis (i.e., not orthogonal to the Z-axis).

FIG. 12 is a schematic view of the source coils 1052a-d with the alternating electrical signals provided in a second combination of phases to generate a second excitation field with a different spatial orientation. In this embodiment, source coils 1052a and 1052c are in phase with each other, and source coils 1052b and 1052d are in phase with each other. However, source coils 1052a and 1052c are 180 degrees out of phase with source coils 1052b and 1052d. The magnetic fields from the source coils 1052a-d combine to generate an excitation field having a magnetic moment 1217 generally in the Y direction within the excitation volume 1109. Accordingly, this excitation field energizes markers parallel to the Y-axis or markers positioned with an angular component along the Y-axis.

FIG. 13 is a schematic view of the source coils 1052a-d with the alternating electrical signals provided in a third combination of phases to generate a third excitation field with a different spatial orientation. In this embodiment, source coils 1052a and 1052b are in phase with each other, and source coils 1052c and 1052d are in phase with each other. However, source coils 1052a and 1052b are 180 degrees out of phase with source coils 1052c and 1052d. The magnetic fields from the source coils 1052a-d combine to generate an excitation field having a magnetic moment 1319 in the excitation volume 1109 generally in the direction of the X-axis. Accordingly, this excitation field energizes markers parallel to the X-axis or markers positioned with an angular component along the X-axis.

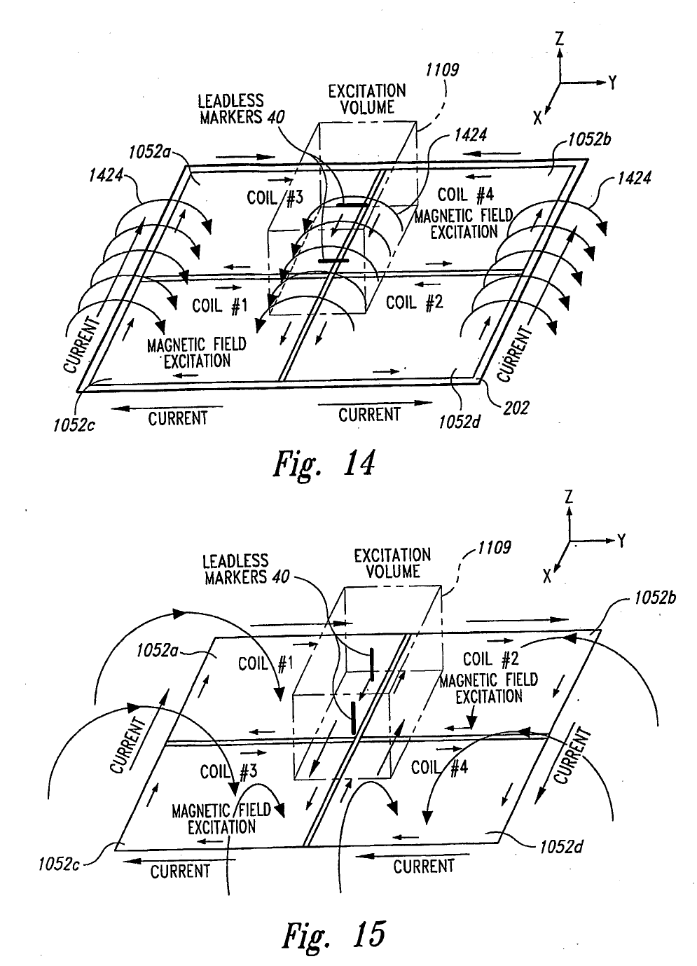

FIG. 14 is a schematic view of the source coils 1052a-d illustrating the current flow to generate an excitation field 1424 for energizing markers 40 with longitudinal axes parallel to the Y-axis. The switching network 1044 (FIG. 10) is configured so that the phases of the alternating electrical signals provided to the source coils 1052a-d are similar to the configuration of FIG. 12. This generates the excitation field 1424 with a magnetic moment in the Y direction to energize the markers 40.

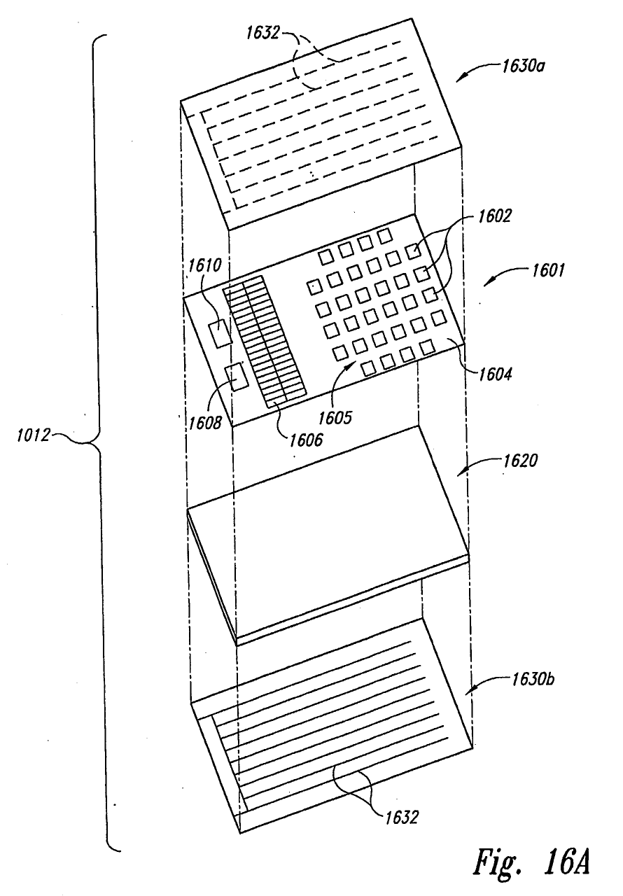

FIG. 15 further illustrates the ability to spatially adjust the excitation field in a manner that energizes any of the markers 40 at different spatial orientations. In this embodiment, the switching network 1044 (FIG. 10) is configured so that the phases of the alternating electrical signals provided to the source coils 1052a-d are similar to the configuration shown in FIG. 11. This produces an excitation field with a magnetic moment in the Z direction that energizes markers 40 with longitudinal axes parallel to the Z-axis.

The spatial configuration of the excitation field in the excitation volume 1109 can be quickly adjusted by manipulating the switching network to change the phases of the electrical signals provided to the source coils 1052a-d. As a result, the overall magnetic excitation field can be changed to be oriented in either the X, Y or Z directions within the excitation volume 1109. This adjustment of the spatial orientation of the excitation field reduces or eliminates blind spots in the excitation volume 1109. Therefore, the markers 40 within the excitation volume 1109 can be energized by the source coils 1052a-d regardless of the spatial orientations of the leadless markers.

In one embodiment, the excitation source 1010 is coupled to the sensor assembly 1012 so that the switching network 1044 (FIG. 10) adjusts orientation of the pulsed generation of the excitation field along the X, Y, and Z axes depending upon the strength of the signal received by the sensor assembly. If the location signal from a marker 40 is insufficient, the switching network 1044 can automatically change the spatial orientation of the excitation field during a subsequent pulsing of the source coils 1052a-d to generate an excitation field with a moment in the direction of a different axis or between axes. The switching network 1044 can be manipulated until the sensor assembly 1012 receives a sufficient location signal from the marker.

The excitation source 1010 illustrated in FIG. 10 alternately energizes the source coils 1052a-d during an excitation phase to power the markers 40, and then actively de-energizes the source coils 1052a-d during a sensing phase in which the sensor assembly 1012 senses the decaying location signals wirelessly transmitted by the markers 40. To actively energize and de-energize the source coils 1052a-d, the switching network 1044 is configured to alternatively transfer stored energy from the energy storage device 1042 to the source coils 1052a-d, and to then re-transfer energy from the source coils 1052a-d back to the energy storage device 1042. The switching network 1044 alternates between first and second “on” positions so that the voltage across the source coils 1052 alternates between positive and negative polarities. For example, when the switching network 1044 is switched to the first “on” position, the energy in the energy storage device 1042 flows to the source coils 1052a-d. When the switching network 1044 is switched to the second “on” position, the polarity is reversed such that the energy in the source coils 1052a-d is actively drawn from the source coils 1052a-d and directed back to the energy storage device 1042. As a result, the energy in the source coils 1052a-d is quickly transferred back to the energy storage device 1042 to abruptly terminate the excitation field transmitted from the source coils 1052a-d and to conserve power consumed by the energy storage device 1042. This removes the excitation energy from the environment so that the sensor assembly 1012 can sense the location signals from the markers 40 without interference from the significantly larger excitation energy from the excitation source 1010. Several additional details of the excitation source 1010 and alternate embodiments are disclosed in U.S. patent application Ser. No. 10/213,980 filed on Aug. 7, 2002, which is incorporated by reference herein in its entirety.

b. Sensor Assemblies

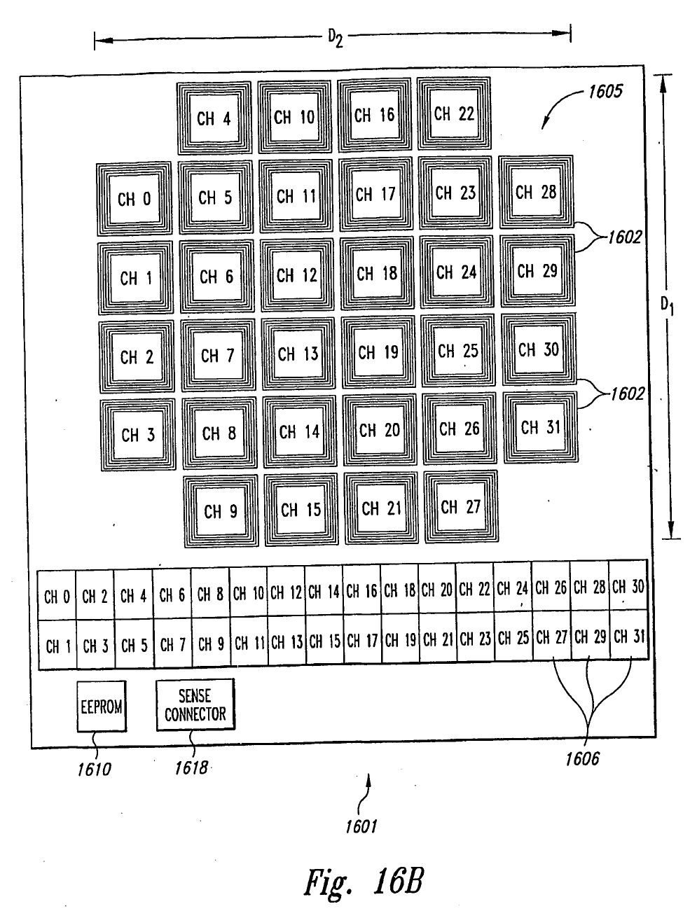

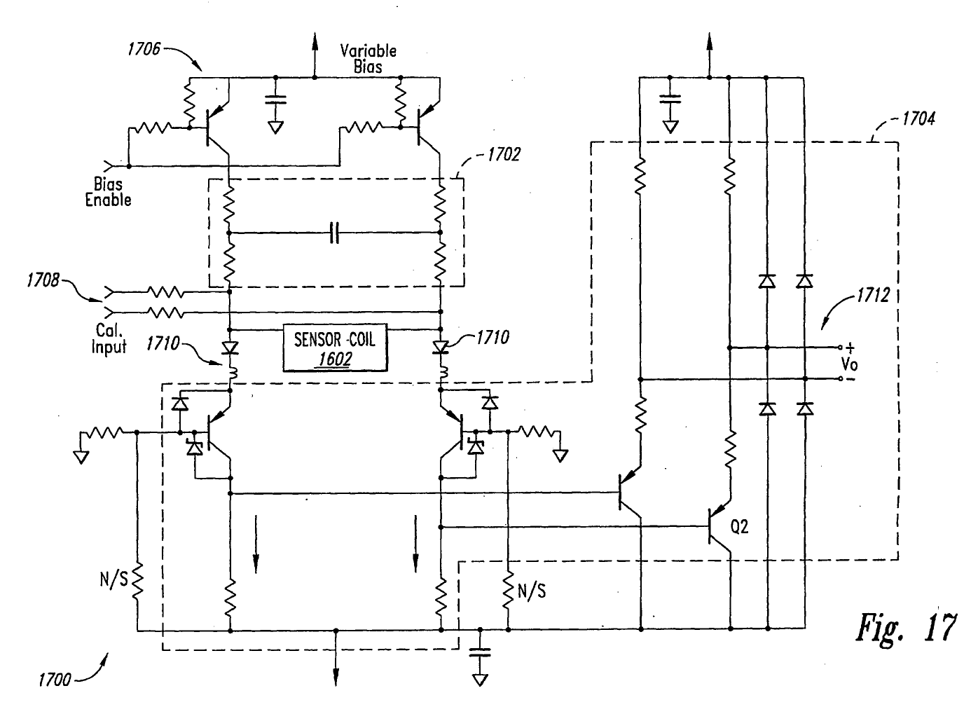

FIG. 16A is an exploded isometric view showing several components of the sensor assembly 1012 for use in the localization system 1000 (FIG. 10). The sensor assembly 1012 includes a sensing unit 1601 having a plurality of coils 1602 formed on or carried by a panel 1604. The coils 1602 can be field sensors or magnetic flux sensors arranged in a sensor array 1605.

The panel 1604 may be a substantially non-conductive material, such as a sheet of KAPTON® produced by DuPont. KAPTON® is particularly useful when an extremely stable, tough, and thin film is required (such as to avoid radiation beam contamination), but the panel 1604 may be made from other materials and have other configurations. For example, FR4 (epoxy-glass substrates), GETEK or other Teflon-based substrates, and other commercially available materials can be used for the panel 1604. Additionally, although the panel 1604 may be a flat, highly planar structure, in other embodiments, the panel may be curved along at least one axis. In either embodiment, the field sensors (e.g., coils) are arranged in a locally planar array in which the plane of one field sensor is at least substantially coplanar with the planes of adjacent field sensors. For example, the angle between the plane defined by one coil relative to the planes defined by adjacent coils can be from approximately 0° to 10°, and more generally is less than 5°. In some circumstances, however, one or more of the coils may be at an angle greater than 10° relative to other coils in the array.

The sensor assembly 1012 shown in FIG. 16A can optionally include a core 1620 laminated to the panel 1604. The core 1620 can be a support member made from a rigid material, or the core 1620 can be a low density foam, such as a closed-cell Rohacell foam. The core 1620 is preferably a stable layer that has a low coefficient of thermal expansion so that the shape of the sensor assembly 1012 and the relative orientation between the coils 1602 remain within a defined range over an operating temperature range.

The sensor assembly 1012 can further include a first exterior cover 1630a on one side of the sensing subsystem and a second exterior cover 1630b on an opposing side. The first and second exterior covers 1630a-b can be thin, thermally stable layers, such as Kevlar or Thermount films. Each of the first and second exterior covers 1630a-b can include electric shielding 1632 to block undesirable external electric fields from reaching the coils 1602. The electric shielding 1632, for example, prevents or minimizes the presence of eddy currents caused by the coils 1602 or external magnetic fields. The electric shielding 1632 can be a plurality of parallel legs of gold-plated, copper strips to define a comb-shaped shield in a configuration commonly called a Faraday shield. It will be appreciated that the shielding can be formed from other Materials that are suitable for shielding. The electric shielding can be formed on the first and second exterior covers using printed circuit board manufacturing technology or other techniques.

The panel 1604 with the coils 1602 is laminated to the core 1620 using a pressure sensitive adhesive or another type of adhesive. The first and second exterior covers 1630a-b are similarly laminated to the assembly of the panel 1604 and the core 1620. The laminated assembly forms a rigid structure that fixedly retains the arrangement of the coils 1602 in a defined configuration over a large operating temperature range. As such, the sensor assembly 1012 does not substantially deflect across its surface during operation. The sensor assembly 1012, for example, can retain the array of coils 1602 in the fixed position with a deflection of no greater than ±0.5 mm, and in some cases no more than ±0.3 mm. The stiffness of the sensing subsystem provides very accurate and repeatable monitoring of the precise location of leadless markers in real time.

In still another embodiment, the sensor assembly 1012 can further include a plurality of source coils that are a component of the excitation source 1010. One suitable array combining the sensor assembly 1012 with source coils is disclosed in U.S. patent application Ser. No. 10/334,700, entitled PANEL-TYPE SENSOR/SOURCE ARRAY ASSEMBLY, filed on Dec. 30, 2002, which is herein incorporated by reference.

FIG. 16B further illustrates an embodiment of the sensing unit 1601. In this embodiment, the sensing unit 1601 includes 32 sensor coils 1602; each coil 1602 is associated with a separate channel 1606 (shown individually as channels “Ch 0” through “Ch 31”). The overall dimension of the panel 1604 can be approximately 40 cm by 54 cm, but the array 1605 has a first dimension D₁ of approximately 40 cm and a second dimension D₂ of approximately 40 cm. The array 1605 can have other sizes or other configurations (e.g., circular) in alternative embodiments. Additionally, the array 1605 can have more or fewer coils, such as 8-64 coils; the number of coils may moreover be a power of 2.

The coils 1602 may be conductive traces or depositions of copper or another suitably conductive metal formed on the panel 1604. Each coil 1602 has a trace with a width of approximately 0.15 mm and a spacing between adjacent turns within each coil of approximately 0.13 mm. The coils 1602 can have approximately 15 to 90 turns, and in specific applications each coil has approximately 40 turns. Coils with less than 15 turns may not be sensitive enough for some applications, and coils with more than 90 turns may lead to excessive voltage from the source signal during excitation and excessive settling times resulting from the coil's lower self-resonant frequency. In other applications, however, the coils 1602 can have less than 15 turns or more than 90 turns.

As shown in FIG. 16B, the coils 1602 are arranged as square spirals, although other configurations may be employed, such as arrays of circles, interlocking hexagons, triangles, etc. Such square spirals utilize a large percentage of the surface area to improve the signal to. noise ratio. Square coils also simplify design layout and modeling of the array compared to circular coils; for example, circular coils could waste surface area for linking magnetic flux from the markers 40. The coils 1602 have an inner dimension of approximately 40 mm, and an outer dimension of approximately 62 mm, although other dimensions are possible depending upon applications. Sensitivity may be improved with an inner dimension as close to an outer dimension as possible given manufacturing tolerances. In several embodiments, the coils 1602 are identical to each other or at least configured substantially similarly.

The pitch of the coils 1602 in the array 1605 is a function of, at least in part, the minimum distance between the marker and the coil array. In one embodiment, the coils are arranged at a pitch of approximately 67 mm. This specific arrangement is particularly suitable when the wireless markers 40 are positioned approximately 7-27 cm from the sensor assembly 1012. If the wireless markers are closer than 7 cm, then the sensing subsystem may include sensor coils arranged at a smaller pitch. In general, a smaller pitch is desirable when wireless markers are to be sensed at a relatively short distance from the array of coils. The pitch of the coils 1602, for example, is approximately 50%-200% of the minimum distance between the marker and the array.

In general, the size and configuration of the array 1605 and the coils 1602 in the array depend on the frequency range in which they are to operate, the distance from the markers 40 to the array, the signal strength of the markers, and several other factors. Those skilled in the relevant art will readily recognize that other dimensions and configurations may be employed depending, at least in part, on a desired frequency range and distance from the markers to the coils.

The array 1605 is sized to provide a large aperture to measure the magnetic field emitted by the markers. It can be particularly challenging to accurately measure the signal emitted by an implantable marker that wirelessly transmits a marker signal in response to a wirelessly transmitted energy source because the marker signal is much smaller than the source signal and other magnetic fields in a room (e.g., magnetic fields from CRTs, etc.). The size of the array 1605 can be selected to preferentially measure the near field of the marker while mitigating interference from far field sources. In one embodiment, the array 1605 is sized to have a maximum dimension D₁ or D₂ across the surface of the area occupied by the coils that is approximately 100% to 300% of a predetermined maximum sensing distance that the markers are to be spaced from the plane of the coils. Thus, the size of the array 1605 is determined by identifying the distance that the marker is to be spaced apart from the array to accurately measure the marker signal, and then arrange the coils so that the maximum dimension of the array is approximately 100% to 300% of that distance. The maximum dimension of the array 1605, for example, can be approximately 200% of the sensing distance at which a marker is to be placed from the array 1605. In one specific embodiment, the marker 40 has a sensing distance of 20 cm and the maximum dimension of the array of coils 1602 is between 20 cm and 60 cm, and more specifically 40 cm.

A coil array with a maximum dimension as set forth above is particularly useful because it inherently provides a filter that mitigates interference from far field sources. As such, one aspect of several embodiments of the invention is to size the array based upon the signal from the marker so that the array preferentially measures near field sources (i.e., the field generated by the marker) and filters interference from far field sources.

The coils 1602 are electromagnetic field sensors that receive magnetic flux produced by the wireless markers 40 and in turn produce a current signal representing or proportional to an amount or magnitude of a component of the magnetic field through an inner portion or area of each coil. The field component is also perpendicular to the plane of each coil 1602. Each coil represents a separate channel, and thus each coil outputs signals to one of 32 output ports 1606. A preamplifier, described below, may be provided at each output port 1606. Placing preamplifiers (or impedance buffers) close to the coils minimizes capacitive loading on the coils, as described herein. Although not shown, the sensing unit 1601 also includes conductive traces or conductive paths routing signals from each coil 1602 to its corresponding output port 1606 to thereby define a separate channel. The ports in turn are coupled to a connector 1608 formed on the panel 1604 to which an appropriately configured plug and associated cable may be attached.

The sensing unit 1601 may also include an onboard memory or other circuitry, such as shown by electrically erasable programmable read-only memory (EEPROM) 1610. The EEPROM 1610 may store manufacturing information such as a serial number, revision number, date of manufacture, and the like. The EEPROM 1610 may also store per-channel calibration data, as well as a record of run-time. The run-time will give an indication of the total radiation dose to which the array has been exposed, which can alert the system when a replacement sensing subsystem is required.

Although shown in one plane only, additional coils or electromagnetic field sensors may be arranged perpendicular to the panel 1604 to help determine a three-dimensional location of the wireless markers 40. Adding coils or sensors in other dimensions could increase the total energy received from the wireless markers 40, but the complexity of such an array would increase disproportionately. The inventors have found that three-dimensional coordinates of the wireless markers 40 may be found using the planar array shown in FIG. 16A-B.

Implementing the sensor assembly 1012 may involve several considerations. First, the coils 1602 may not be presented with an ideal open circuit. Instead, they may well be loaded by parasitic capacitance due largely to traces or conductive paths connecting the coils 1602 to the preamplifiers, as well as a damping network (described below) and an input impedance of the preamplifiers (although a low input impedance is preferred). These combined loads result in current flow when the coils 1602 link with a changing magnetic flux. Any one coil 1602, then, links magnetic flux not only from the wireless marker 40, but also from all the other coils as well. These current flows should be accounted for in downstream signal processing.

A second consideration is the capacitive loading on the coils 1602. In general, it is desirable to minimize the capacitive loading on the coils 1602. Capacitive loading forms a resonant circuit with the coils themselves, which leads to excessive voltage overshoot when the excitation source 1010 is energized. Such a voltage overshoot should be limited or attenuated with a damping or “snubbing” network across the coils 1602. A greater capacitive loading requires a lower impedance damping network, which can result in substantial power dissipation and heating in the damping network.

Another consideration is to employ preamplifiers that are low noise. The preamplification can also be radiation tolerant because one application for the sensor assembly 1012 is with radiation therapy systems that use linear accelerators (LINAC). As a result, PNP bipolar transistors and discrete elements may be preferred. Further, a DC coupled circuit may be preferred if good settling times cannot be achieved with an AC circuit or output, particularly if analog to digital converters are unable to handle wide swings in an AC output signal.

FIG. 17, for example, illustrates an embodiment of a snubbing network 1702 having a differential amplifier 1704. The snubbing network 1702 includes two pairs of series coupled resistors and a capacitor bridging therebetween. A biasing circuit 1706 allows for adjustment of the differential amplifier, while a calibration input 1708 allows both input legs of the differential amplifier to be balanced. The coil 1602 is coupled to an input of the differential amplifier 1704, followed by a pair of high voltage protection diodes 1710. DC offset may be adjusted by a pair of resistors coupled to bases of the input transistors for the differential amplifier 1704 (shown as having a zero value). Additional protection circuitry is provided, such as ESD protection diodes 1712 at the output, as well as filtering capacitors (shown as having a 10 nF value).

C. Signal Processors and Controllers

The signal processor 1014 and the controller 1016 illustrated in FIG. 10 receive the signals from the sensor assembly 1012 and calculate the absolute positions of the markers 40 within the reference frame. Suitable signal processing systems and algorithms are set forth in U.S. application Ser. Nos. 10/679,801; 10/749,478; 10/750,456; 10/750,164; 10/750,165; 10/749,860; and 10/750,453, all of which are incorporated herein by reference.

From the foregoing, it will be appreciated that specific embodiments of the invention have been described herein for purposes of illustration, but that various modifications may be made without deviating from the spirit and scope of the invention. Accordingly, the invention is not limited except. as by the appended claims.

I/We claim:

1. An instrument for stimulating and/or sensing neurons in the nervous system of a patient, comprising:
a body configured to be implanted into a patient;
an electrode contact carried by the body and an electrically conductive line coupled to the electrode contact; and
a marker carried by the body, the marker having a transponder configured to be energized by a wirelessly transmitted excitation energy and to wirelessly transmit a location signal in response to the excitation energy.

2. The instrument of claim 1 wherein the body comprises a shaft configured to be implanted into a subdural region of the brain of the patient, and the electrode contact comprises an electrically conductive member exposed along a portion of the shaft.

3. The instrument of claim 2 wherein the electrode contact comprises a band around a portion of the shaft.

4. The instrument of claim 1 further comprising a plurality of electrode contacts including a first electrode contact at a first location on the body and a second electrode contact at a second location on the body spaced apart from the first location.

5. The instrument of claim 1 wherein the body comprises a shaft having a distal section configured to be implanted at a subdural location in the brain of the patient, and wherein the instrument further comprises a plurality of electrode contacts including a first electrode contact at a first location on the distal section of the body and a second electrode contact at a second location on the distal section of the body spaced apart from the first location.

6. The instrument of claim 5 wherein the first and second electrode contacts are coupled to a common lead to be biased at the same potential.

7. The instrument of claim 5 wherein the first electrode contact is coupled to a first lead and the second electrode contact is coupled to a second lead such that first and second electrode contacts can be biased at different potentials.

8. The instrument of claim 1 wherein the transponder comprises an alternating magnetic circuit having a ferrite core and a coil with a plurality of windings around the ferrite core.

9. The instrument of claim 1 wherein the transponder comprises a ferrite core and a coil around the ferrite core, and wherein the marker further comprises a capsule encasing the transponder, the capsule having a longitudinal axis and a cross-sectional dimension normal to the longitudinal axis of not greater than 2 mm.

10. The instrument of claim 1 wherein the marker comprises a capsule and the transponder comprises an alternating magnetic circuit within the capsule, and wherein the transponder is not electrically coupled to external leads outside of the capsule.

11. The instrument of claim 1 wherein the marker comprises a capsule and an alternating magnetic circuit in the capsule, and wherein the marker has a radiographic centroid and the alternating magnetic circuit has a magnetic centroid at least approximately coincident with the radiographic centroid.

12. The instrument of claim 1 wherein the marker comprises an alternating magnetic circuit having a ferrite core, a coil having a plurality of windings around the core, and an imaging element, and wherein the marker has a radiographic centroid and the alternating magnetic circuit has a magnetic centroid at least approximately coincident with the radiographic centroid.

13. The instrument of claim 1 wherein the marker comprises an alternating magnetic circuit having a ferrite core extending along a longitudinal axis, a coil having a plurality of windings around the core, and a capsule encasing the core and the coil, and wherein the core has a maximum cross-sectional dimension normal to the longitudinal axis of not greater than 0.7 mm and the capsule has a maximum cross-sectional dimension normal to the longitudinal axis of not greater than 2 mm.

14. The instrument of claim 1, further comprising a drug delivery element along the body.

15. An instrument for stimulating and/or sensing neurons in the nervous system of a patient, comprising:
an elongated shaft configured to be implanted into a patient;
an electrode contact carried by the shaft and an electrically conductive line coupled to the electrode contact; and
a marker attached to the shaft, the marker having an alternating magnetic circuit configured to be energized by a wirelessly transmitted pulsed magnetic excitation field and to wirelessly transmit a pulsed magnetic location signal in response to the magnetic excitation field.

16. The instrument of claim 15 wherein the marker comprises a capsule encasing the alternating magnetic circuit, and wherein the marker has a radiographic centroid and the alternating magnetic circuit has a magnetic centroid at least approximately coincident with the radiographic centroid.

17. The instrument of claim 15 wherein the alternating magnetic circuit comprises a ferrite core, a coil having a plurality of windings around the core, and an imaging element, and wherein the marker has a radiographic centroid and the alternating magnetic circuit has a magnetic centroid at least approximately coincident with the radiographic centroid.

18. The instrument of claim 15 wherein the alternating magnetic circuit comprises a ferrite core extending along a longitudinal axis, a coil having a plurality of windings around the core, and a capsule encasing the core and the coil, and wherein the core has a maximum cross-sectional dimension normal to the longitudinal axis of not greater than 0.7 mm and the capsule has a maximum cross-sectional dimension normal to the longitudinal axis of not greater than 2 mm.

19. An electrode for subdural sensing and/or stimulation in a brain of a patient, comprising:
an elongated body having a distal section configured to be implanted at a subdural location in the brain of the patient and a proximal section;
a lead connector at the proximal section of the body;
an electrode contact on the distal section of the body;
an electrical conductor coupled to the electrode contact and the lead connector; and
a marker carried by the body at a fixed location with respect to the electrode contact, the marker comprising an alternating magnetic transponder configured to be energized by a wirelessly transmitted excitation energy and produce a wirelessly transmitted location signal in response to the excitation energy.

20. The electrode of claim 19 wherein the marker comprises a capsule encasing the alternating magnetic transponder, and wherein the marker has a radiographic centroid and the alternating magnetic transponder has a magnetic centroid at least approximately coincident with the radiographic centroid.

21. The electrode of claim 19 wherein the alternating magnetic transponder comprises a ferrite core, a coil having a plurality of windings around the core, and an imaging element, and wherein the marker has a radiographic centroid and the alternating transponder has a magnetic centroid at least approximately coincident with the radiographic centroid.

22. The electrode of claim 19 wherein the alternating magnetic transponder comprises a ferrite core extending along a longitudinal axis, a coil having a plurality of windings around the core, and a capsule encasing the core and the coil, and wherein the core has a maximum cross-sectional dimension normal to the longitudinal axis of not greater than 0.7 mm and the capsule has a maximum cross-sectional dimension normal to the longitudinal axis of not greater than 2 mm.

23. A stimulation system, comprising:
an implantable stimulus unit having an energy source and a pulse generator coupled to the energy source for providing an electrical stimulation waveform;
a stimulation lead configured to be coupled to the implantable stimulus unit, the simulation lead having a flexible dielectric cover and a conductor within the cover, and the simulation lead being configured to be implanted within the patient; and
an instrument having a body configured to be implanted into a patient, an electrode contact carried by the body and configured to be electrically coupled to the stimulation lead for delivering the stimulation waveform to the patient, and a marker carried by the body, wherein the marker comprises a transponder configured to be energized by a wirelessly transmitted excitation energy and to wirelessly transmit a location signal in response to the excitation energy.

24. The system of claim 23 wherein the body comprises a shaft configured to be implanted into a subdural region of the brain of the patient, and the electrode contact comprises an electrically conductive member exposed along a portion of the shaft.

25. The system of claim 24 wherein the electrode contact comprises a band around a portion of the shaft.

26. The system of claim 23 further comprising a plurality of electrode contacts on the body, the electrode contacts including a first electrode contact at a first location on the body and a second electrode contact at a second location on the body spaced apart from the first location.

27. The system of claim 23 wherein the body comprises a shaft having a distal section configured to be implanted at a subdural location in the brain of the patient, and wherein the instrument further comprises a plurality of electrode contacts including a first electrode contact at a first location on the distal section of the body and a second electrode contact at a second location on the distal section of the body spaced apart from the first location.

28. The system of claim 27 wherein the first and second electrode contacts are coupled to a common lead to be biased at the same potential.

29. The system of claim 27 wherein the first electrode contact is coupled to a first lead and the second electrode contact is coupled to a second lead such that first and second electrode contacts can be biased at different potentials.

30. The system of claim 23 wherein the transponder comprises an alternating magnetic circuit having a ferrite core and a coil with a plurality of windings around the ferrite core.

31. The system of claim 23 wherein the transponder comprises a ferrite core and a coil around the ferrite core, and wherein the marker further comprises a capsule encasing the transponder, the capsule having a longitudinal axis and a cross-sectional dimension normal to the longitudinal axis of not greater than 2 mm.

32. The system of claim 23 wherein the marker comprises a capsule and the transponder comprises an alternating magnetic circuit within the capsule, and wherein the transponder is not electrically coupled to external leads outside of the capsule.

33. The system of claim 23 wherein the marker comprises a capsule and an alternating magnetic circuit in the capsule, and wherein the marker has a radiographic centroid and the alternating magnetic circuit has a magnetic centroid at least approximately coincident with the radiographic centroid.

34. The system of claim 23 wherein the marker comprises an alternating magnetic circuit having a ferrite core, a coil having a plurality of windings around the core, and an imaging element, and wherein the marker has a radiographic centroid and the alternating magnetic circuit has a magnetic centroid at least approximately coincident with the radiographic centroid.

35. The system of claim 23 wherein the marker comprises an alternating magnetic circuit having a ferrite core extending along a longitudinal axis, a coil having a plurality of windings around the core, and a capsule encasing the core and the coil, and wherein the core has a maximum cross-sectional dimension normal to the longitudinal axis of not greater than 0.7 mm and the capsule has a maximum cross-sectional dimension normal to the longitudinal axis of not greater than 2 mm.

36. The system of claim 23 wherein the instrument further comprises a drug delivery element along the body.

37. A stimulation system, comprising:
an implantable stimulus unit having an energy source and a pulse generator coupled to the energy source for providing an electrical stimulation waveform;
a stimulation lead configured to be coupled to the implantable stimulus unit, the simulation lead having a flexible dielectric cover and a conductor within the cover, and the simulation lead being configured to be implanted within the patient; and
an instrument having an elongated body including a distal section configured to be implanted at a subdural location in the brain of the patient and a proximal section configured to be connected to the stimulation lead, an electrode contact on the distal section of the body for delivering the stimulation waveform to the patient, and a marker carried by the body at a fixed location with respect to the electrode contact, the marker comprising a leadless alternating magnetic transponder configured to be energized by a wirelessly transmitted excitation energy and to wirelessly transmit a location signal in response to the excitation energy.

38. A system for sensing and/or stimulating a population neurons in the central nervous system of a patient, comprising:
an instrument having a body configured to be implanted into a patient, an electrode contact carried by the body, and a marker carried by the body, wherein the marker comprises a transponder having a circuit configured to be energized by a wirelessly transmitted pulsed magnetic excitation field and to wirelessly transmit a pulsed location signal in response to the pulsed magnetic excitation field; and
an excitation source comprising an energy storage device, a source coil, and a switching network coupled to the energy storage device and the source coil, the source coil being configured to wirelessly transmit the pulsed magnetic excitation field to energize the transponder, and the switching network being configured to alternately transfer (a) stored energy from the energy storage device to the source coil and (b) energy in the source coil back to the energy storage device.

39. The system of claim 38 wherein the switching network comprises an H-bridge switch.

40. The system of claim 38 wherein the switching network is configured to have a first on position in which the stored energy is transferred from the energy storage device to the source coil and a second on position in which energy in the source coil is transferred back to the energy storage device.

41. The system of claim 40 wherein the first on position has a first polarity and the second on position has a second polarity opposite the first polarity.

42. The system of claim 38 wherein the source coil comprises an array having a plurality of substantially coplanar coils.

43. The system of claim 42 wherein the switching network is configured to selectively energized the coplanar coils to change a spatial configuration of the pulsed magnetic field.

44. The system of claim 38 wherein the body comprises a shaft configured to be implanted into a subdural region of the brain of the patient, and the electrode contact comprises an electrically conductive member exposed along a portion of the shaft.

45. The system of claim 38 wherein the electrode contact comprises a band around a portion of the shaft.

46. The system of claim 38 further comprising a plurality of electrode contacts on the body, the electrode contacts including a first electrode contact at a first location on the body and a second electrode contact at a second location on the body spaced apart from the first location.

47. The system of claim 38 wherein the body comprises a shaft having a distal section configured to be implanted at a subdural location in the brain of the patient, and wherein the instrument further comprises a plurality of electrode contacts including a first electrode contact at a first location on the distal section of the body and a second electrode contact at a second location on the distal section of the body spaced apart from the first location.

48. The system of claim 38 wherein the first and second electrode contacts are coupled to a common lead to be biased at the same potential.

49. The system of claim 38 wherein the first electrode contact is coupled to a first lead and the second electrode contact is coupled to a second lead such that first and second electrode contacts can be biased at different potentials.

50. The system of claim 38 wherein the circuit comprises an alternating magnetic circuit having a ferrite core and a coil with a plurality of windings around the ferrite core.

51. The system of claim 38 wherein the circuit comprises a ferrite core and a coil around the ferrite core, and wherein the marker further comprises a capsule encasing the transponder, the capsule having a longitudinal axis and a cross-sectional dimension normal to the longitudinal axis of not greater than 2 mm.

52. The system of claim 38 wherein the marker comprises a capsule and the circuit comprises an alternating magnetic circuit within the capsule, and wherein the transponder is not electrically coupled to external leads outside of the capsule.

53. The system of claim 38 wherein the marker comprises a capsule and the circuit is in the capsule, and wherein the marker has a radiographic centroid and the alternating magnetic circuit has a magnetic centroid at least approximately coincident with the radiographic centroid.

54. The system of claim 38 wherein the circuit comprises an alternating magnetic circuit having a ferrite core, a coil having a plurality of windings around the core, and an imaging element, and wherein the marker has a radiographic centroid and the alternating magnetic circuit has a magnetic centroid at least approximately coincident with the radiographic centroid.

55. The system of claim 38 wherein the circuit comprises an alternating magnetic circuit having a ferrite core extending along a longitudinal axis, a coil having a plurality of windings around the core, and a capsule encasing the core and the coil, and wherein the core has a maximum cross-sectional dimension normal to the longitudinal axis of not greater than 0.7 mm and the capsule has a maximum cross-sectional dimension normal to the longitudinal axis of not greater than 2 mm.

56. The system of claim 38 wherein the instrument further comprises a drug delivery element along the body.

57. A system for sensing and/or stimulating a population neurons in the central nervous system of a patient, comprising:
an instrument having a body configured to be implanted into a patient, an electrode contact carried by the body, and a marker carried by the body, wherein the marker comprises a transponder having a circuit configured to be energized by a wirelessly transmitted pulsed excitation field and to wirelessly transmit a pulsed location signal in response to the pulsed excitation field; and
a sensing assembly comprising a support member and a plurality of field sensors carried by the support member, the field sensors being at least substantially locally planar relative to one another and configured to sense the pulsed location signal from the marker.

58. The system of claim 57 wherein the field sensors are responsive only to field components of the location signal normal to individual field sensors.

59. The system of claim 57 wherein the field sensors are arranged in an array occupying an area having a maximum dimension of approximately 100% to 300% of a predetermined sensing distance between the marker and the sensing array.

60. The system of claim 57 wherein the body comprises a shaft configured to be implanted into a subdural region of the brain of the patient, and the electrode contact comprises an electrically conductive member exposed along a portion of the shaft.

61. The system of claim 57 further comprising a plurality of electrode contacts on the body, the electrode contacts including a first electrode contact at a first location on the body and a second electrode contact at a second location on the body spaced apart from the first location.

62. The system of claim 57 wherein the first and second electrode contacts are coupled to a common lead to be biased at the same potential.

63. The system of claim 57 wherein the first electrode contact is coupled to a first lead and the second electrode contact is coupled to a second lead such that first and second electrode contacts can be biased at different potentials.

64. The system of claim 57 wherein the circuit comprises an alternating magnetic circuit having a ferrite core and a coil with a plurality of windings around the ferrite core.

65. The system of claim 57 wherein the circuit comprises a ferrite core and a coil around the ferrite core, and wherein the marker further comprises a capsule encasing the transponder, the capsule having a longitudinal axis and a cross-sectional dimension normal to the longitudinal axis of not greater than 2 mm.

66. The system of claim 57 wherein the marker comprises a capsule and the circuit comprises an alternating magnetic circuit within the capsule, and wherein the transponder is not electrically coupled to external leads outside of the capsule.

67. The system of claim 57 wherein the marker comprises a capsule and the circuit is in the capsule, and wherein the marker has a radiographic centroid and the alternating magnetic circuit has a magnetic centroid at least approximately coincident with the radiographic centroid.

68. The system of claim 57 wherein the circuit comprises an alternating magnetic circuit having a ferrite core, a coil having a plurality of windings around the core, and an imaging element, and wherein the marker has a radiographic centroid and the alternating magnetic circuit has a magnetic centroid at least approximately coincident with the radiographic centroid.

69. The system of claim 57 wherein the circuit comprises an alternating magnetic circuit having a ferrite core extending along a longitudinal axis, a coil having a plurality of windings around the core, and a capsule encasing the core and the coil, and wherein the core has a maximum cross-sectional dimension normal to the longitudinal axis of not greater than 0.7 mm and the capsule has a maximum cross-sectional dimension normal to the longitudinal axis of not greater than 2 mm.

70. The system of claim 57 wherein the instrument further comprises a drug delivery element along the body.

71. A method of implanting an instrument used for sensing and/or stimulating a population of neurons at a selected stimulation site in a patient, comprising:
inserting into the patient an instrument having an electrode contact and a marker including a transponder; and
tracking the instrument in a reference volume when the instrument is in the patient by (a) wirelessly delivering a pulsed excitation signal to energize the transponder, (b) wirelessly transmitting a pulsed location signal from the transponder to a location outside of the patient, (c) sensing the pulsed location signal at a sensor located outside of the patient, and (d) calculating the location of the marker in the three-dimensional reference volume.

72. The method of claim 71 wherein inserting the instrument into the patient comprises moving the instrument through the brain to a deep brain location, and tracking the instrument comprises periodically calculating the location of the marker in the reference volume while moving the instrument through the brain.

73. The method of claim 71 wherein inserting the instrument into the patient comprises moving the instrument through the brain to a deep brain location, and tracking the instrument comprises (a) periodically calculating a location of the marker in the reference volume while moving the instrument through the brain, and (b) periodically determining a relative offset between the electrode contact and the stimulation site based on the periodically calculated locations of the marker.

74. The method of claim 73, further comprising displaying the relative offset between the electrode contact and the stimulation site.

75. The method of claim 73, further comprising terminating movement of the instrument when the relative offset between the electrode contact and the stimulation site is within a desired range.

76. The method of claim 73, further comprising providing an indication of when the relative offset between the electrode contact and the stimulation site is within an acceptable range.

77. A method for tracking an instrument used for sensing and/or stimulating a population of neurons at a selected stimulation site in a patient, comprising:
implanting an instrument into the patient, the instrument having an electrode contact and a marker including a transponder;
tracking the instrument with respect to the stimulation site by (a) wirelessly delivering a pulsed excitation signal to energize the transponder, (b) wirelessly transmitting a location signal from the transponder to a location outside of the patient, (c) sensing the pulsed location signal at a sensor located outside of the patient, and (d) periodically calculating the location of the marker in a reference volume; and
providing an output of the location of the marker in the reference volume at least every t_{f} seconds and within tₗ seconds from sensing the location signal, wherein t_{f} and tₗ are not greater than 1 second.

78. The method of claim 77 wherein tf and tl are from approximately 10 ms to approximate 500 ms

79. The method of claim 77 wherein tf and tl are from approximately 20 ms to approximate 200 ms

80. The method of claim 77 wherein tf and tl are from approximately 50 ms to approximate 200 ms

81. The method of claim 77 wherein tf and tl are from approximately 50 ms to approximate 100 ms

82. The method of claim 77 wherein implanting the instrument into the patient comprises moving the instrument through the brain to a deep brain location, and tracking the instrument comprises periodically calculating the location of the marker in the reference volume while moving the instrument through the brain.

83. The method of claim 77 wherein implanting the instrument into the patient comprises moving the instrument through the brain to a deep brain location, tracking the instrument comprises periodically calculating the location of the marker in the reference volume while moving the instrument through the brain, and providing an output of the location of the marker comprises providing a relative offset between the electrode contact and the stimulation site based on the periodically calculated locations of the marker.

84. The method of claim 83, further comprising displaying the relative offset between the electrode contact and the stimulation site.

85. The method of claim 83, further comprising terminating movement of the instrument when the relative offset between the electrode contact and the stimulation site is within a desired range.

86. The method of claim 83, further comprising providing an indication of when the relative offset between the electrode contact and the stimulation site is within an acceptable range.

87. A method for implanting an instrument for sensing and/or stimulating a population of neurons at a selected stimulation site in a patient, comprising:
implanting into the patient an instrument having an electrode contact and a marker including a transponder;
determining the location of the instrument in a reference volume by (a) wirelessly delivering a pulsed excitation signal to energize the transponder, (b) wirelessly transmitting a pulsed location signal from the transponder to a location outside of the patient, (c) sensing the pulsed location signal at a sensor located outside of the patient, and (d) calculating the location of the marker in a three-dimensional reference volume; and
receiving electrical signals at the electrode contact from the population of neurons and/or delivering electrical stimulation from the electrode contact.
